SEQUENCE LISTING (1 sequence)

SEQ ID NO: 1 - protein, 109 aa, Homo sapiens
NLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

(19) United States
(12) Patent Application Publication
(10) Pub. No.: US 2023/0263860 A1
(43) Pub. Date: Aug. 24, 2023

(54) Methods And Compositions For Treating Stroke

(71) Applicant: ELEVIAN, INC., Miami Beach, FL (US)

(72) Inventors: Yongting Wang, Lexington, MA (US); Mark Felder Allen, Menlo Park, CA (US); Anthony Sandrasagra, Arlington, MA (US); Manisha Sinha, Newton, MA (US)

(21) Appl. No.: 17/926,551

(22) PCT Filed: May 19, 2021
(86) PCT No.: PCT/US2021/033241

§ 371 Date: Nov. 18, 2022

Related U.S. Application Data
(60) Provisional application No. 63/026,809, filed on May 19, 2020.

(51) Int. Cl. A61K 38/18; A61P 25/28; A61P 9/10

(52) U.S. Cl. CPC A61K 38/1875; A61P 9/10; A61P 25/28

(57) ABSTRACT

Compositions and methods for treating stroke in a mammalian subject are provided. The compositions and methods generally entail administration of an effective amount of a growth and differentiation factor 11 (GDF 11) molecule to a subj ect initiating within a short time window after a stroke event, wherein the GDF 11 molecule is administered at a high dose relative to the body weight of the subject and such administration regimen is carried out over a short period of time ranging from 1 to about 14 days.

### PRIORITY

This patent application claims the benefit under 35 U.S.C. §119(e) of U.S. Pat. Application Serial No. 63/026,809, entitled “Methods and Compositions for Treating Stroke,” filed on May 19, 2020, which is incorporated herein by reference in its entirety.

### TECHNICAL FIELD

The present disclosure generally relates to methods and compositions for treating stroke in a subject featuring administration of a growth differentiation factor 11 molecule (GDF11) to that subject.

### INTRODUCTION

A number of studies have shown that certain blood-borne factors play a major role in regulating tissue homeostasis and regeneration (see, e.g., Conboy et al. (2005) Nature 433(7027): 760-764; Ruckh et al. (2012) Cell Stem Cell 10(1): 96-103; Loffredo et al. (2013) Cell 153(4): 828-839; Katsimpardi et al. (2014) Science 344(6184): 630-634; and Sinha et al. (2014) Science 344(6184): 649-652). One such circulating blood factor is growth differentiation factor (GDF11), which has been demonstrated to broadly and consistently stimulate regenerative capacity in multiple different tissue systems including skin and muscle, cardiovascular, and neurological (see, e.g., International Patent Publication Nos.: WO 2013/142114; WO 2014/168973; WO 2014/201143; and WO 2015/070076). Subsequent to the above-noted publications, the following ubiquitous therapeutic and regenerative effects of GDF11 have been recognized and reported: improved tubular regeneration after kidney ischemia-reperfusion injury (Zhang et al (2016) Scientific Reports 6(1): 34624); protection against endothelial injury (Mei et al. (2016) Molecular Therapy: the Journal of the American Society of Gene Therapy 24(11): 1926-1938); enhanced myocardial regeneration after cardiac ischemia-reperfusion injury (Du et al. (2017) Basic Research in Cardiology 112(1): 7); improved insulin secretion and glucose tolerance in type 2 diabetes (Li et al. (2017) Diabetes 66(7): db170086-1927); improved lung function in emphysema (COPD) (Onodera et al. (2017) Thorax April); tumor-suppression in triple-negative breast cancer (Bajikar et al. (2017) Developmental Cell 43(4): 418-435); reversal of cardiac hypertrophy (Harper et al. (2018) Circulation Research 7 Sep. 2018); promotion of neovascularization and blood flow in diabetic limb ischemia (Zhang et al. (2018) Diabetes); improved neurobehavioral recovery and angiogenesis in cerebral ischemia-reperfusion injury (Ma et al. (2018) Brain Research Bulletin 139(February): 38-47); rescued cognitive function and improved cerebrovascular function in Alzheimer’s Disease (Zhang et al. (2018) Journal of Alzheimer’s Disease 62(2)); promotion of cerebral neovascularization, neurogenesis and functional recovery after stroke (Lu et al. (2018) Front Cell Neurosci 12: 205); prevention of intestinal inflammosome activation during ulcerative colitis (Wang et al. (2018) Am J Physiology Gastrointest Liver Physiology); and attenuated skin inflammation in psoriasis (Wang et al. (2018) Inflammation).

### SUMMARY

In 2013, approximately 6.9 million people had an ischemic stroke, and 3.4 million people had a hemorrhagic stroke (Global Burden of Disease Study 2013 Collaborators (August 2015) The Lancet 386(9995): 743-800). In 2015, stroke was the second most frequent cause of death after coronary artery disease, accounting for 11% of the total (GBD 2015 Mortality and Causes of Death Collaborators (October 2016) The Lancet 388(10053): 1459-1544). It is generally accepted that a vast majority of stokes are ischemic since it is difficult to ascertain if a hemorrhagic stroke was initiated by an ischemic event. Current medical intervention and treatment of stroke is exceptionally time-sensitive. In acute ischemic stroke, the gold standard of thrombolysis, such as with recombinant tissue plasminogen activator (rtPA), when administered within three hours of symptom onset provides an overall benefit of about 10% with respect to living without disability but does not improve chances of survival (see, e.g., Wardlaw et al. (July 2014) The Cochrane Database of Systemic Reviews 7(7): CD000213 and Emberson et al. (2014) The Lancet 384(9958): 1929-1935). The desirability of administration of thrombolytics between three and four and a half to five hours of symptom onset is subject to debate as regards to whether it provides therapeutic benefit or causes potential further damage. Intra-arterial fibrinolysis, where a catheter is passed up an artery into the brain and medication is injected at the site of thrombosis has shown benefit in improving outcomes in acute ischemic stroke (see, e.g., Lee et al. (2010) Stroke 41(5): 932-937). Mechanical removal of a blood clot causing ischemic stroke (mechanical thrombectomy) represents another potential treatment for occlusion of a large artery, e.g., the middle cerebral artery), and published reviews have reported the safety and efficacy of such procedures in reducing disability if performed within up to 24 hours of the onset of symptoms, but again without improving chances of survival (see, e.g., Sardar et al. (2015) European Heart Journal 36(35): 2373-2380; Saver et al. (2016) JAMA 316(12): 1279-1288; Goyal et al. (2016) The Lancet 387(10029): 1723-1731; Mistry et al. (2017) Stroke 48(9): 2450-2456; and Powers et al. (2018) Stroke 49(3): e46-e110).

Given the low therapeutic benefit of medicinal interventions administered quickly after a stroke event, treatment of stroke has evolved into a traditional three-pronged approach, where the first prong consists of prevention. For example, in a population that have had a myocardial infarction or other high cardiovascular risk, daily aspirin may have certain preventative effects. For the population that has previously had a stroke, treatment with aspirin, clopidogrel and dipyridamole may provide benefit. Modifiable risk factors such as high blood pressure, atrial fibrillation, high cholesterol levels, diabetes mellitus and the like can be likewise treated using methods well known in the medicinal arts. The second prong of treatment entails the medicinal and mechanical approaches detailed above that are applied in the time-window that occurs immediately after a stroke event arises. The third prong entails rehabilitation, for example physical therapy, occupational therapy and speech-language pathology. In this regard, although stroke survivors typically show some degree of functional recovery within the first few months, they are often left with significant neurological deficits including motor, sensory, and cognitive impairment. Although there is a generally held belief that little can be done to replace nerve cells and circuits that have been permanently lost due to stroke, a number of approaches have been reviewed that seek to effect an emerging fourth prong of stroke therapeutics, that is, the possible medicinal repair or rejuvenation of neurological tissue and systems damaged by stroke (see, e.g., Iaci et al. (2013) Stroke 44: 1942-1950 (dalfampridine) and Iaci et al. (2016) Journal of Neuroscience Research 94: 253-265 (Neuregulin 1ß3, Glial Growth Factor)). However, to date, there are no approved pharmaceuticals for repair or rejuvenation of neurological tissue and/or systems damaged by a stroke event.

There accordingly exists an urgent and long-felt need in the art to develop and employ novel methods and compositions suitable for repair or rejuvenation of neurological tissue and/or systems damaged by a stroke event. In answer to this urgent and long-felt need, the present inventors have found, surprisingly, that administration of high doses of a growth differentiation factor 11 (GDF11) molecule to a subject within from about 12 hours to about 3 days after a stroke event in the subject, over a limited administration period, brings about durable and sustained treatment (repair or rejuvenation of neurological tissue and/or systems damaged by a stroke event) without concomitant adverse effects.

Multiple studies have reported neovascularization and proliferation of endothelial cells upon daily administration of rGDF11 in rodent stroke, diabetes related peripheral artery disease, and AD models (see, e.g., Ma et al. (2018) Brain Research Bulletin 139(February): 38-47; Lu et al. (2018) Front Cell Neurosci 12: 205; and Zhang et al. (2018) Journal of Alzheimer’s Disease 62(2)). In particular, in an Alzheimer’s disease (AD) mouse model, daily intravenous administration of rGDF11 for 28 days at 0.1 mg/kg has been shown to improve cerebrovascular structure, function, blood flow and also rescue cognitive function (Zhang et al. (2018), id.). Studies in young adult rat cerebral ischemia/reperfusion (I/R) model demonstrated that daily intravenous dosing of rGDF11 at 0.1 mg/kg daily for 7 or 14 days post I/R improved neurofunctional recovery, increased the number of functional microvessels in the peri-infarct cortex and promoted the proliferation of brain endothelial cells and neuronal regeneration (Ma et al. (2018), id.). Likewise, intraperitoneal administration of rGDF11 at 0.1 mg/kg daily for 7 days in young adult mice post I/R resulted in enhanced neurofunctional recovery, neurogenesis, and neuronal regeneration starting at 14 days (Lu et al. 2018), id.). Furthermore, in a stroke model using aged mice, it was observed that injection of rGDF11 at 0.1 mg/kg daily for 5 days starting at day-5 after ischemic stroke resulted in significantly improved mortality rate (See Chauhan et al. (2018) Stroke 49). Other benefits of rGDF11 treatment in this aged murine stroke model include reduced gliosis, increased angiogenesis, restored white matter integrity, and synaptic plasticity in addition to the reduction of brain tissue loss and pathological lateral ventricle enlargement, increased number of NeuN+ neurons and improved motor functions at Day 14 and Day 30 after stroke (Chauhan et al. (2018), id.).

More recently, it was reported that daily injection of rGDF11 for 28 days prior to experimental intracerebral hemorrhage (ICH) reduced neurological deficits and alleviated ICH-induced edema, inflammation, apoptosis, oxidative stress, and mitochondria damage in the brain perihematomal tissue of 24-month aged rats (See, Anqi et al. (2019) J Clin Neurosci 63: 182-188). Pretreatment with lentivirus carrying the GDF11 gene was shown to be protective against cerebral I/R in a rat model (see Zhao et al. (2020) Brain Res. 1737: 146802). The GDF11-lentivirus pretreatment reduced cerebral infarction volume and apoptotic cells and promoted behavioral recovery as well as promoted neurogenesis and angiogenesis in the subventricular zone (SVZ). It was also reported that a single injection of 1.25 ng of rGDF11 into the lateral ventricle at 24 hours after reperfusion reduced brain infarction volume, reduced number of apoptotic cells in the peri-infarct cerebral cortex and promoted behavioral recovery at 5 days after transient ischemic stroke (Zhao et al. (2020), id.).

It is notable that all of the above-referenced studies targeted low or so-called “moderate” dose administration of GDF11 (either a single administration of 1.25 ng of GDF11, or daily dosing of GDF11 at 0.1 mg/kg in a rodent subject). The present inventors believe this is due to two considerations. First, it is believed that baseline GDF11 levels in mammals is about 3-5 ng/mL (unpublished), and thus past dosing paradigms have been structured to increase GDF11 plasma levels incrementally rather than substantially. Second, and perhaps more significantly, a number of studies have reported that supraphysiological or so-called “excess” doses of GDF11 give rise to significant adverse effects including cachexia, muscle atrophy, anorexia and renal fibrosis (see, e.g., Hammers et al. (2017) EMBO Molecular Medicine 9(4): 531-544; Pons et al. (2018) Surgery (May); and Jones et al. (2018) Cell Reports 22(6): 1522-1530). In addition, increased GDF11 levels have been found in colorectal cancer (Gu et al. (2018) Cell Mol Biology Noisy-le-grand France 64: 80-84). Although a single study in the aging hippocampus reported daily administration of GDF11 at doses up to 1 mg/kg (Ozek et al. (2018) Sci Rep 8: 17293) without observation of significant adverse effects, there certainly exists a strong bias in the art against targeting the use of high dose GDF11 in any contemplated treatment.

Accordingly, it is a primary aspect of the present disclosure to provide a method for treating stroke in a subject. The method entails beginning a dosing regimen by administering a therapeutically effective amount of a GDF11 molecule to the subject within the time frame of about 12 to 72 hours after a stroke event in the subject. The GFD11 molecule is administered in an amount of at least about the minimal high dose of GDF11 relative to the body weight of the subject and is carried out over a period of from 2 to about 14 days. In one aspect of the disclosure, the GDF11 molecule is administered over a period of from 2 to about 7 days. In one particular aspect of the present disclosure, the method entails initiation of the dosing regimen within about 12 to 24 hours after the stroke event. In another particular aspect, administration of the GDF11 molecule is carried out over a period of from 2 to 4 days. In yet another aspect of the disclosure, the GDF11 molecule is administered to the subject in an amount of from about 1 to 2 mg/kg per day in a rodent subject or at the corresponding dose in a larger mammalian subject. Practice of the methods of the present disclosure, that is, rapid initiation of the dosing regimen (within from about 12 hours to about 3 days from the stroke event), high dose administration of the GDF11 molecule, and limited duration of treatment (from 2 to about 14 days) is effective to provide durable and sustained treatment in the stroke subject without concomitant adverse effects.

The GDF11 molecule used in the practice of the present methods is any therapeutically active form of a GDF11 molecule that can be the same or different in the compositions employed over the course of the dosing regimen. In this regard, native GDF11 protein in humans is encoded by the GDF11 gene and has a molecular structure that is identical in humans, mice and rats. The sequence of GDF11 is thus highly conserved across several mammalian species, and GDF11 is known to be expressed in many tissues, including skeletal muscle, pancreas, kidney, the nervous system and retina. For human GDF11, the pro-peptide plus signal sequence (e.g. the precursor polypeptide) is 407 amino acids long. Cleavage of the 24 amino acid signal peptide generates a pro-peptide of 383 amino acids and cleavage of the pro-peptide results in a mature GDF11 polypeptide of 109 amino acids that corresponds to the C-terminal 109 amino acids of the pro-peptide. The mature form of GDF11 polypeptide molecule forms a disulfide-linked homodimer. Accordingly, any derivative, variant or modified form of a “native” GDF11 molecule can be determined to be a “therapeutically active” GDF11 molecule by comparison of the pharmacological activity of the subject molecule against the activity of the mature form of the native human GDF11 polypeptide (in its fully active form as a homodimer) using methods and techniques well known to those of ordinary skill in the art.

Accordingly, in certain preferred aspects of the present disclosure, the GDF11 molecule selected for use in the methods can be a mature form of a GDF11 polypeptide that can be provided in the form of a homodimer. The selected GDF11 molecule can alternatively be a polypeptide with at least 91% sequence homology with the native sequence of human GDF11. Preferably, the GDF11 molecule is recombinant human GDF11 (rhGDF11) in the mature form. In other aspects, the GDF11 molecule is a therapeutically active variant or derivative of the human GDF11 molecule. Such variant or derivative can include one or more amino acid substitutions or deletions relative to the native sequence of the human GDF11 molecule and can include one or more amino acid analog. In yet other aspects of the disclosure, the selected GD11 molecule can be a modified GDF11 polypeptide, for example, where the molecule has been phosphorylated, glycated, glycosylated, pegylated, HESylated, ELPylated, lipidated, acetylated, amidated, end-capped, includes a cyano group or albumin, or is cyclized. Alternatively, the modified GDF11 molecule can be a chimeric polypeptide having at least two moieties, a first GDF11 molecule moiety and a second moiety, for example, where the second moiety is derived from transferrin, growth hormone or an Fc fragment. In preferred aspects, the modified GDF11 molecule will have an increased half-life relative to the mature form of native GD11 polypeptide. In any event, the selected GDF11 molecule is formulated in a suitable pharmaceutical composition for administration to the subject that can further include a pharmaceutically acceptable carrier, excipient or vehicle.

Administration of the GDF11 molecule is carried out via parenteral administration since GDF11 does not appear to cross the blood brain barrier (BBB) and is efficacious when made available systemically. Administration of the GDF11 molecule can further be carried out on a once daily (QD) basis, twice daily (BID), three times daily (TID), four times daily (QID), hourly (“q_h” where “h” denotes the number of hours between doses), or the like, and each day of treatment can be the same or different over the course of treatment. In a particular aspect of the disclosure, administration is carried out once daily (QD). For example, a composition containing the GDF11 molecule can be administered to a subject intravenously by way of a catheter such as a central venous catheter line or like IV catheter. Alternatively, the GDF11 composition can be administered via intravenous, intramuscular, intraperitoneal or subcutaneous injection using a standard needle and syringe. In certain aspects, the composition can thus be simply formulated to include a suitable injection vehicle such as water for injection. In yet other aspects, the composition can be administered using an external drug pump such as an infusion pump. The compositions can further include a controlled, sustained or delayed release excipient. In the practice of such methods, the composition can be provided in the form of a nanoparticle such as a liposome. The compositions can further include a bioerodible polymer or nonpolymer controlled release excipient and can be provided in the form of an injectable liquid implant or in the form of a microparticle. In the practice of the methods of the present disclosure, the GDF11 molecule can be present in the composition in the form of a solution, suspension or emulsion.

In a particular aspect of the present disclosure, the precise dosage and duration of treatment employed in the practice of the methods is a function of the type of stroke and resulting stroke damage that is being addressed and can be determined empirically using known testing protocols or by extrapolation from in vivo or in vitro test data or subsequent clinical testing. It is to be noted that concentrations and dosage values can also vary with the severity of the stroke to be addressed. For any particular subject, specific dosage regimens can be adjusted over time according to the individual need and the professional judgment of the person administering or supervising the administration of the present GDF11 compositions and the concentration ranges set forth herein are exemplary only and are not intended to limit the scope or practice of the claimed methods. In other aspects of the disclosure, administration of the compositions can be carried out once, or can entail any number of treatment regimens suitable for the specific treatment being contemplated. For example, a suitable treating regimen can include a first administration (on day one of the treatment) of the GDF11 molecule at a first dose followed by a second administration or subsequent administrations of the GDF11 molecule at a second or more higher or lower dose (e.g., on day 2 up to day 7 of the treatment). In certain preferred aspects, the dosing regimen entails classical titration of the GDF 11 molecule in either ascending or descending doses, for example wherein the first administration is carried out at an initial dose of at least about the minimal high dose of GDF11 relative to the body weight of the subject on day 1 of the treatment period and finishes at a second, higher dose. Alternatively, titration of the GDF11 molecule can entail an initial (day one) high dose of the GDF11 molecule and ending with a final dose of at least about the minimal high dose of GDF11 in the subject. In any titration strategy, it may be preferred to administer the GDF11 molecule at a first high dose approaching the median toxic dose (MTD) for that molecule, or at least approaching the maximum dose of the therapeutic window for the administered GDF11 molecule, followed by a subsequent dose (or doses) at lower level. In other aspects of the disclosure, the GDF11 treatment regimens can be carried out multiple times (e.g., repeated), with a so-called “drug holiday”, that is, by following a structured treatment interruption, tolerance break or treatment break, e.g., where subsequent treatment(s) occur from 2 to 7 days after completion of the initial treatment. Here again, for any particular subject, specific dosing regimens can be adjusted over time according to the individual need and the professional judgment of the person administering or supervising the administration of the present GDF11 compositions and the dosing strategies set forth herein are exemplary only and are not intended to limit the scope or practice of the claimed methods.

Successful conduct of the present methods can be assessed using diagnostic and clinical examination techniques well known in the art. For example, such techniques are typically based on physical and neurological examination (such as the NIHSS) to assess improved body motor function and/or cognitive function in the subject, and can be supported by medical imaging techniques such as CT scan, MRI scan, Doppler ultrasound and arteriography and often supported by ancillary tests such as electrocardiogram (ECG) and blood tests. Medical imaging techniques can assess successful stroke treatment in a subject by way of visualizing neovascularization, neurogenesis, improved cerebrovascular structure, and/or function or blood flow at or near the site of stroke in a subject, for example in the stroke penumbra where blood flow has been reduced locally near the location of the original insult (ischemia). For clarity, successful stroke therapy using the methods of the present disclosure can be established by assessing any one or more (and any combination thereof) of the above-noted criteria and/or by employing any one or more of the above-noted diagnostic and imaging techniques.

These aspects of the present disclosure, as well as others, are described in detail in the following sections of this application and expressly set forth in the appended claims.

### BRIEF DESCRIPTION OF THE FIGURES

FIG. 1 depicts results from one prong of the middle cerebral artery occlusion (MCAO) study described in Example 1, wherein therapeutic efficacy of administration of rGDF11 was assessed in a body swing (behavioral) test. Animals receiving GDF11 at 1 mg/kg QD showed superior stroke recovery compared to placebo (vehicle only) animals on Day 7 (p<0.001), Day 14 (p<0.001), Day 21 (p<0.001) and Day 28 (p<0.001). n=24 rats.

FIG. 2 depicts results from a second prong of the MCAO study described in Example 1, wherein therapeutic efficacy of administration of rGDF11 was assessed in a hindlimb placing (behavioral) test. Animals receiving GDF11 at 1 mg/kg QD showed superior stroke recovery compared to placebo (vehicle only) animals on Day 7 (p<0.001), Day 14 (p<0.001), Day 21 (p<0.001) and Day 28 (p<0.001). n=24 rats.

FIG. 3 depicts results from a third prong of the MCAO study described in Example 1, wherein therapeutic efficacy of administration of rGDF11 was assessed in a forelimb placing (behavioral) test. Animals receiving GDF11 at 1 mg/kg QD showed superior stroke recovery compared to placebo (vehicle only) animals on Day 14 (p<0.05) and a trend for Day 21 (p=0.60). n=24 rats.

FIG. 4 depicts results from the fourth prong of the MCAO study described in Example 1, wherein the side effect profile of administration of rGDF11 was assessed. Animals receiving rGDF11 at 1 mg/kg showed significant reduction of weight compared to the vehicle-treated animals (p<0.001). Body weight in the rGDF11 treated animals was reduced by 7.03% on the third day post-surgery relative to their weights immediately after surgery. Thereafter, rGDF11 treated animals gained weight at a rate comparable to the vehicle treated animals. n=24 rats.

FIG. 5 depicts results from one prong of the MCAO study described in Example 2, wherein therapeutic efficacy of administration of rGDF11 was assessed in a forelimb placing (behavioral) test. Animals that received rGDF11 i.p. at 1 mg/kg, on Day 1 through Day 7, showed superior recovery compared to vehicle-treated animals on Day 3 (p<0.0001), Day 7 (p<0.001), Day 14 (p<0.001), Day 21 (p<0.0001), and Day 30 (p<0.001).

FIG. 6 depicts results from one prong of the MCAO study described in Example 2, wherein therapeutic efficacy of administration of rGDF11 was assessed in a hindlimb placing (behavioral) test. Animals that received rGDF11 i.p. at 1 mg/kg, at Day 1 through Day 7, showed superior recovery compared to vehicle-treated animals on Day 3 (p<0.001), Day 5 (p<0.01), Day 7 (p<0.0001), Day 14 (p<0.001), Day 21 (p<0.0001), and Day 30 (p<0.0001).

FIG. 7 depicts results from one prong of the MCAO study described in Example 2, wherein therapeutic efficacy of administration of rGDF11 was assessed in a body swing (behavioral) test. Animals that received rGDF11 i.p. at 1 mg/kg, at Day 3 through Day 16, showed superior recovery compared to vehicle-treated animals on Day 7 (p<0.05), Day 14 (p<0.001), Day 21 (p<0.001), and Day 30 (p<0.01).

FIG. 8 depicts results from one prong of the MCAO study described in Example 3, wherein therapeutic efficacy of administration of rGDF11 was assessed in a forelimb placing (behavioral) test. Animals that received rGDF11 i.p. at 1 mg/kg daily for 3 Days, 5 Days, and 7 Days showed statistically improved recovery compared to vehicle-treated animals 14 days after stroke (p<0.05 for 3 days of treatment, p<0.0001 for 5 days of treatment, p<0.001 for 7 days of treatment).

FIG. 9 depicts results from one prong of the MCAO study described in Example 3, wherein therapeutic efficacy of administration of rGDF11 was assessed in a hindlimb placing (behavioral) test. Animals that received rGDF11 i.p. at 1 mg/kg daily for 1 Day, 3 Days, 5 Days, and 7 Days showed statistically improved recovery compared to vehicle-treated animals 14 days after stroke (p<0.05 for 1 days of treatment; p<0.001 for 3 days of treatment, p<0.0001 for 5 days of treatment, p<0.0001 for 7 days of treatment).

FIG. 10 depicts results from one prong of the MCAO study described in Example 3, wherein therapeutic efficacy of administration of rGDF11 was assessed in a body swing (behavioral) test. Animals that received rGDF11 i.p. at 1 mg/kg daily for 5 Days and 7 Days showed statistically improved recovery compared to vehicle-treated animals 14 days after stroke (p<0.001 for 5 days of treatment, p<0.0001 for 7 days of treatment).

FIG. 11 depicts results from one prong of the MCAO study described in Example 4, wherein therapeutic efficacy of administration of rGDF11 was assessed in a forelimb placing (behavioral) test. Animals that received rGDF11 i.p. doses of 1 mg/kg, 2 mg/kg, and 4 mg/kg showed statistically improved recovery compared to vehicle-treated animals 28 days after stroke (p<0.05 for 1 mg/kg, p<0.001 for 2 mg/kg, p<0.001 for 4 mg/kg).

FIG. 12 depicts results from one prong of the MCAO study described in Example 4, wherein therapeutic efficacy of administration of rGDF11 was assessed in a hindlimb placing (behavioral) test. Animals that received rGDF11 i.p. doses of 1 mg/kg, 2 mg/kg, and 4 mg/kg showed statistically improved recovery compared to vehicle-treated animals 28 days after stroke (p<0.001 for 1 mg/kg, p<0.0001 for 2 mg/kg, p<0.0001 for 4 mg/kg).

FIG. 13 depicts results from one prong of the MCAO study described in Example 4, wherein therapeutic efficacy of administration of rGDF11 was assessed in a body swing (behavioral) test. Animals that received rGDF11 i.p. doses of 0.5 mg/kg, 1 mg/kg, 2 mg/kg, and 4 mg/kg showed statistically improved recovery compared to vehicle-treated animals 28 days after stroke (p<0.001 for 0.5 mg/kg, p<0.001 for 1 mg/kg, p<0.0001 for 2 mg/kg, p<0.0001 for 4 mg/kg).

FIG. 14 depicts the percent survival of Group A (rGDF11 treatment group; 1 mg/kg, dosed once daily for seven (7) days) and Group B (vehicle group) for C57B16/j mice as a function of days post-ICH. The vehicle group showed a trend toward increased mortality as compared to the rGDF11 treatment group.

FIG. 15A depicts the post-ICH NeuroSeverity Score over the days Group A and Group B following injection, including dead animals. The vehicle group showed increased NeuroSeverity as compared to the rGDF11 treatment group at 28 days (p<0.05).

FIG. 15B depicts the post-ICH NeuroSeverity Score over the days Group A and Group B following injection, excluding dead animals. The vehicle group showed increased NeuroSeverity as compared to the rGDF11 treatment group at 28 days (p<0.01).

FIG. 16A depicts a plot of rotarod latency as a function of days post-ICH injury for Group A and the Group B, including dead animals. Group A showed improvement in rotarod latency compared to Group B at 28 days (p<0.05).

FIG. 16B depicts a plot of rotarod latency as a function of days post-ICH injury for Group A and the Group B, excluding dead animals. Group A showed improvement in rotarod latency compared to Group B at 28 days (p<0.0001).

FIG. 17A shows the average speed in in centimeters per second seven (7) days after first treatment (p<0.05).

FIG. 17B depicts the forelimb base of support at seven (7) days after first treatment for the rGDF11 administered group (Group A) compared to the vehicle group (Group B) (p<0.05).

FIGS. 18A - 18D depict replenishment of progenitor cells in the subventricular zone ipsilateral to the injury site: FIG. 18A (Vehicle), FIG. 18B (1 mg/kg), FIG. 18C (2 mg/kg), and FIG. 18D (4 mg/kg) at Day 29.

FIG. 19 depicts the increase in neurogenesis for 1 mg/kg dose, 2 mg/kg dose, and 4 mg/kg dose of GDF11 in the subventricular zone ipsilateral to the injury site compared to vehicle-treated animals at Day 29.

FIGS. 20A - 20D depict replenishment of progenitor cells in the subventricular zone contralateral to the injury site: FIG. 20A (Vehicle), FIG. 20B (1 mg/kg), FIG. 20C (2 mg/kg), and FIG. 20D (4 mg/kg)) at Day 29.

FIG. 21 depicts the increase in neurogenesis for 1 mg/kg dose, 2 mg/kg dose, and 4 mg/kg dose of GDF11 in the subventricular zone contralateral to the injury site.

FIG. 22 depicts an analysis comparing hemispheres ipsilateral and contralateral to the injury site.

### DETAILED DESCRIPTION

For convenience, certain terms employed in this entire application (including the specification, figures and appended claims) are expressly defined throughout. Unless expressly defined otherwise, all technical and scientific terms used herein have the same meaning as commonly understood by one of ordinary skill in the art to which this disclosure belongs.

### Definitions

As used throughout this specification and the appended claims, the singular forms “a,” “an” and “the” include plural referents unless the content clearly dictates otherwise. Thus, for example, reference to “a pharmaceutically acceptable carrier, excipient or vehicle” includes a mixture of two or more such entities, and the like.

As used herein, the term “comprising” means that other elements can also be present in addition to the defined elements presented. The use of “comprising” therefore indicates inclusion rather than limitation. The term “consisting of” refers to compositions, methods, and respective components thereof as described herein, which are exclusive of any element not recited in that description of the aspect. As used herein the term “consisting essentially of” refers to those elements required for a given aspect. The term permits the presence of elements that do not materially affect the basic and novel or functional characteristic(s) of that aspect of the disclosure.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lan, Antibodies: A Laboratory Manual, Cold Spring Harbor Laboratory Press, Cold Spring Harbor, N.Y. (1990); Principles of Neural Science, 4th ed., Eric R. Kandel, James H. Schwart, Thomas M. Jessell eds. McGraw-Hill/Appleton & Lange: New York, N.Y. (2000); The Merck Manual of Diagnosis and Therapy, 19th Edition, published by Merck Research Laboratories (2006) (ISBN 0-911910-19-0), Robert S. Porter et al. eds., The Encyclopedia of Molecular Biology, published by Blackwell Science Ltd. (1994) (ISBN 0-632-02182-9); and Current Protocols in Protein Sciences (2009) Wiley Intersciences, Coligan et al., eds.

Unless defined otherwise, all technical and scientific terms used herein have the same meaning as commonly understood by one of ordinary skill in the art.

Other than in the operating examples, or where otherwise indicated, all numbers expressing quantities of ingredients or reaction conditions used herein should be understood as modified in all instances by the term “about” whether or not expressly indicated as such. The term “about” when used in connection with percentages, days or dosages or other amounts can mean +/- 10%.

As used herein, the terms “administer”, “administering”, and “administered” refer to providing a therapeutically active agent to the subject being treated. Administration of the GDF11 molecule can be carried out on any suitable basis, such as once daily (QD) basis, twice daily (BID), three times daily (TID), four times daily (QID), hourly (“q_h” where “h” denotes the number of hours between doses), or the like, and each day of treatment can be the same or different over the course of treatment. In certain aspects, the GDF11 molecule is administered in the form of a liquid solution or suspension that is introduced to the subject via normal intraperitoneal, subcutaneous, or intravenous delivery techniques, but can include microinjection, stereotactic injection, and/or direct application to a particular site in the subj ect.

The terms “biodegradable” and “bioerodible” are used interchangeably herein and refer to a material such as a polymer that will degrade or erode over time in vivo to form smaller chemical species, wherein the degradation or erosion can result, for example, from enzymatic, chemical, and physical processes. Most commonly, such materials degrade or erode via hydrolysis to produce degradation products that present no significant, deleterious or untoward effects on a subject’s body. Examples of biodegradable polymers suitable for use in the compositions and methods of the present disclosure include, but are not limited to: poly(lactide)s; poly(glycolide)s; poly(lactide-co-glycolide)s; poly(lactic acid)s; poly(glycolic acid)s; and poly(lactic acid-co-glycolic acid)s; poly(caprolactone)s; poly(malic acid)s; polyamides; polyanhydrides; polyamino acids; polyorthoesters; polyetheresters; polycyanoacrylates; polyphosphazines; polyphosphoesters; polyesteramides; polydioxanones; polyacetals; polyketals; polycarbonates; polyorthocarbonates; degradable polyurethanes; polyhydroxybutyrates; polyhydroxyvalerates; polyalkylene oxalates; polyalkylene succinates; chitins; chitosans; oxidized celluloses; and copolymers, terpolymers, blends, combinations or mixtures of any of the above materials.

The term “controlled release” refers to a pharmaceutical dosage form or composition that provides for the delayed, slowed over a period of time, continuous, discontinuous, or sustained release of a therapeutically active molecule.

The terms “decrease”, “reduce”, “reduced”, “reduction”, “decrease”, and “inhibit” are all used interchangeably herein generally to mean a decrease by a statistically significant amount relative to a reference. However, for avoidance of doubt, such terms typically mean a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The phrase “dose of GDF11” or “dose of GDF11 molecule”, as used interchangeably herein, denotes the quantity of GDF11 administered to a subject over a specified period of time, wherein such quantity is measured as relative to the body weight of the subject being treated. To date, all published reports of actual in vivo dosing of GDF11 have been in rodent (mouse and rat) animal model systems. From these published studies, two distinct dosing paradigms have emerged. The first paradigm, a therapeutic GDF11 strategy, has clearly emerged, and is referred to herein generally as targeting a “moderate dose of GDF11”. This moderate dose strategy includes the vast majority of published in vivo GDF11 dosing studies targeting from about a 1 to 4 fold increase in circulating GDF11 in the treated subject, where mouse or rat subjects are treated with GDF11 administered daily at a typical amount of about 0.1 mg/kg body weight, on a daily basis. See, e.g., Zhang et al (2016) Scientific Reports 6(1): 34624; Mei et al. (2016) Molecular Therapy: the Journal of the American Society of Gene Therapy 24(11): 1926-1938; Du et al. (2017) Basic Research in Cardiology 112(1): 7; Li et al. (2017) Diabetes 66(7): db170086-1927; Onodera et al. (2017) Thorax (April); Bajikar et al. (2017) Developmental Cell 43(4): 418-435; Harper et al. (2018) Circulation Research 7 Sep. 2018; Zhang et al. (2018) Diabetes; Ma et al. (2018) Brain Research Bulletin 139(February): 38-47; Zhang et al. (2018) Journal of Alzheimer’s Disease 62(2); Lu et al. (2018) Front Cell Neurosci 12: 205; Wang et al. (2018) Am J Physiology Gastrointest Liver Physiology; and Wang et al. (2018) Inflammation. Such moderate dose administration of GDF11 has been characterized as extending from 0.001 mg/kg to up to 0.5 mg/kg body weight (see, e.g, U.S. Pat. No. 9,434,779; and U.S. Pat. Publication Nos. US2016/074477 and US2016/220640). Accordingly, as used herein, a “moderate dose of GDF11” shall mean moderate application of a broad range of GDF11 molecule to a subject over the specified period of time, typically in the range of from about 0.001 mg/kg up to about 0.5 mg/kg body weight daily in a subject and preferably at about 0.1 mg/kg body weight daily in a rodent subject and as normally extrapolated to other larger mammalian species up to and including human subjects. As noted above, published reports of in vivo administration of moderate doses of GDF11 have avoided the high end (0.5 mg/kg), and instead concentrated on a specific moderate dose of 0.1 mg/kg as a gold standard. Therapeutic administration of such “moderate doses of GDF11” in these publications has demonstrated therapeutic benefit across a wide spectrum of disease and conditions.

The second GDF11 dosing paradigm, referred to herein as an “excess dose of GDF11” is a dose of GDF11 that, although providing some or no therapeutic benefit, may result in generating adverse or otherwise unacceptable side effect or side effect profile in the subject being treated with GDF11. As used herein, a “side effect” is any pharmacological or physiological effect of GDF11 administration that is secondary to the one intended, and an “adverse side effect” is any such secondary effect that is undesired and/or harmful, and that manifests in an outcome such as morbidity, mortality, loss of function or any other pathological change in a treated subject where it may be reversible or irreversible. A number of published in vivo GDF11 dosing studies targeting excess doses of GDF11, where mouse or rat subjects were treated with GDF11 administered daily at a typical amount of about 5 mg/kg to 10 mg/kg body weight or greater, on a daily basis, have reported that such supraphysiological doses of GDF11 may give rise to significant adverse effects including cachexia, muscle atrophy, anorexia, fibrosis or even death (see, e.g., Hammers et al. (2017) EMBO Molecular Medicine 9(4): 531-544; Pons et al. (2018) Surgery (May); and Jones et al. (2018) Cell Reports 22(6): 1522-1530).

As used herein, “GDF11” or a “GDF11 molecule” refers to “Growth and Differentiation Factor 11” (NCBI Gene ID No: 10220), a member of the Transforming Growth Factor-ß (“TGF-ß”) superfamily of growth factors. GDF11 is known to bind TGF-ß superfamily type I receptors including ALK4, ALK5, and ALK7. The term “rGDF11” refers to an GDF11 molecule that has been produced using recombinant methods, and the term “rhGDF11” refers to such molecules that are derived from the native human GDF11 molecule. rhGDF11 has the amino acid sequence defined in SEQ ID NO: 1. For signaling in mammalian development, GDF11 predominantly uses ALK4 and ALK5. In some aspects, GDF11 signaling can also occur via the ACVR2B receptor. GDF11 is also closely related to Growth and Differentiation Factor 8 (GDF8, also known as myostatin). GDF11 can also be referred to as Bone Morphogenic Protein 11, i.e., BMP11. Accordingly, as used herein, reference to “GDF11” or “GDF11 molecule” includes the human precursor polypeptide (NCBI Ref Seq: NP_005802); the human pro-peptide; the human N-terminal polypeptide, and the human mature forms of GDF11 as well as homologs from other species, including but not limited to bovine, dog, cat chicken, murine, rat, porcine, ovine, turkey, horse, fish, baboon and other primates. The terms also refer to fragments, derivatives or variants of GDF11 that maintain at least 50% of the physiological (therapeutic) effect of the mature GDF11, e.g. as measured in an appropriate animal model. Conservative substitution variants that maintain suitable activity of wildtype GDF11 will include a conservative substitution as defined herein. The identification of amino acids most likely to be tolerant of conservative substitution while maintaining at least 50% of the activity of the wildtype is guided by, for example, sequence alignment with GDF11 homologs or paralogs from other species. Amino acids that are identical between GDF11 homologs are less likely to tolerate change, while those showing conservative differences are obviously much more likely to tolerate conservative change in the context of an artificial variant. Similarly, positions with non-conservative differences are less likely to be critical to function and more likely to tolerate conservative substitution in an artificial variant. Variants can be tested for activity, for example, by administering the variant to a subject in an appropriate animal model as described herein to assess therapeutic efficacy. For human GDF11, the pro-peptide plus signal sequence (e.g. the precursor polypeptide) is 407 amino acids long. Cleavage of the 24 amino acid signal peptide generates a pro-peptide of 383 amino acids and cleavage of the pro-peptide results in a mature GDF11 polypeptide of 109 amino acids that corresponds to the C-terminal 109 amino acids of the pro-peptide. The mature polypeptide forms a disulfide-linked homodimer.

The term “high dose of GDF11” as used herein defines a dose of a GDF11 molecule administered to a subject that falls in between a moderate dose of GDF11 and an excess dose of GDF11 in the relevant subject and that has, surprisingly, demonstrated an unexpected beneficial effect in the treatment of stroke as set forth in this disclosure. A high dose of GDF11 molecule is generally defined herein as encompassing a bracketed range of doses starting from greater than the top end of reported moderate doses of GDF11, to less than the bottom reported excess dose of GDF11, both such moderate and excess doses of GDF11 as reported on a daily basis and in a relevant subject. More particularly, a standard high dose of GDF11 is defined herein as equivalent to about 1 mg/kg body weight of a rodent subject (mouse or rat) on a daily basis, accordingly about 1 order of magnitude greater than the normal moderate dose of GDF11 in such species (i.e., about 0.1 mg/kg). Accordingly, a “minimal high dose of GDF11” is at least about 0.8 mg/kg (body weight) in a rodent species, and the same such dose in a larger mammalian species, normalized to the molecular weight of rhGDF11 as defined by SEQ ID NO: 1. In like manner, a “maximal high dose of GDF11” is about 4 mg/kg (body weight) in a rodent species and the same such dose in a larger mammalian species, normalized to the molecular weight of rhGDF11 as defined by SEQ ID NO: 1. In this regard, the maximal high dose of GDF11 is that which avoids adverse side effects in the treated subject. It is to be noted that specific dosage values can vary with the severity of the stroke to be addressed. For any particular subject, specific dosage regimens can be adjusted over time according to the individual need and the professional judgment of the person administering or supervising the administration of the present GDF11 compositions and the concentration ranges set forth herein are exemplary only and are not intended to limit the scope or practice of the claimed methods. Exact dosing can be predicted empirically by testing in in vitro and in vivo systems well known to those of skill in the art and then extrapolated therefrom for use in subjects including human subjects. Human doses are then typically fine-tuned in clinical trials and titrated to response. For the purposes of this disclosure, a high dose of GDF11, as defined in a rodent subject, is at least about 0.8 mg/kg to about 4 mg/kg, or at least about 0.9 mg/kg to about 4 mg/kg, in some cases at least about 1 mg/kg to about 3 mg/kg, and in certain aspects of the disclosure at least about 1 to 2 mg/kg, and encompasses the corresponding dose in any larger mammalian species.

As used herein, the term “hydrogel” is used in its usual manner within the art, for example to refer to a polymer that swells in the presence of water or other aqueous system, shrinks in the absence or reduction of the amount of water, is able to retain a significant fraction of water within its structure, and typically does not dissolve in water. One ordinarily skilled in the art will appreciate that there are a number of standard tests that one can employ in order to determine if a polymer or polymer system will act as a hydrogel, e.g., form a hydrogel, when immersed in an aqueous system such as when it is implanted or otherwise delivered in vivo into a mammalian subject.

An “implant” or “implantable composition or device”, as used herein, refers to any implantable system for use in the delivery of a therapeutically active substance to a subject. Common implantable devices allow local (site specific) and/or systemic administration of the agent of interest and examples include solid structures such as stents or wafers that can be left behind on or in tissue at a surgical site, rods or microparticles that can be administered via subcutaneous or intramuscular injection with a needle and syringe or trocar, and implantable drug pump devices. Solid implantable compositions or devices are commonly formed using bioerodible polymers that can provide for controlled release of an agent of interest. Injectable implantable compositions or devices can be provided in the form of viscous liquid carriers, hydrogel compositions, nanoparticle compositions, microspheres or microparticles, or plasticized polymer carriers.

The terms “increased”, “increase” or “enhanced” are all used interchangeably herein generally to mean an increase by a statically significant amount; for the avoidance of any doubt, the terms denote an increase of at least 10% as compared to a reference level, for example an increase of at least about 20%, or at least about 30%, or at least about 40%, or at least about 50%, or at least about 60%, or at least about 70%, or at least about 80%, or at least about 90%, or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or more as compared to a reference level.

The term, “kit” as used herein, means any manufacture (e.g., a package or container) including at least one therapeutically active agent (a GDF11 molecule). In certain kits the manufacture may be promoted, distributed, or sold as a unit for performing the methods of the present disclosure.

As used herein, the term “liposome” refers to a spherical vesicle having at least one lipid bilayer formed by certain lipids (phospholipids such as phosphatidylcholine) that are filled with an aqueous core that may contain other components including a therapeutically active agent of the present disclosure. A liposome can be up to about 10 microns in size, however, preferably the liposomes employed in the practice of the compositions and methods of the present disclosure are sub-micron in size, i.e., in the form of a nanoparticle.

The term “local” or “locally” as used herein means, with respect to delivery or administration of a therapeutically active agent to a subject, that such agent is delivered to a localized site in the subject but may not be detectable at a biologically significant level in the blood plasma of the subject.

A “nanoparticle” refers to a particulate material or a population of such particles with sizes generally ranging between 1 and 100 nm and can include nanospheres, for example lipid systems such as liposomes and micelles, nanocrystals and nanoparticles. Nanospheres can contain pharmacological agents (molecules or compounds) as well as other materials such as inorganic nanoparticles like gold or magnetic particles, or nanoparticles may contain or be formed from polymers such as biodegradable polymers. In the practice of the compositions and methods of the present disclosure, synthetic polymers such as polyvinyl alcohol, poly-L-lactic acid, polyethylene glycol and poly(lactic-co-glycolic acid and natural polymers such as alginate and chitosan can be used in the nanofabrication of nanoparticles to provide nanospheres or nanocapsules. Nanoparticles generally remain in the blood circulatory system for a prolonged period, thus enabling the extended release of agents and extended pharmacological agent life cycle. Due to their nanosize, nanoparticle structures readily penetrate tissue systems and facilitate easy uptake by cells to achieve efficient delivery at targeted locations.

The term “pharmaceutically acceptable” refers to a material that has been approved or is approvable for pharmaceutical use by a regulatory agency of a relevant federal or state government and/or is listed in the U.S. Pharmacopeia or another generally recognized pharmacopeia for use in animal subjects, and more particularly in humans. A “pharmaceutically acceptable carrier, excipient or vehicle” refers to any vehicle, diluent, adjuvant, excipient or carrier with which a therapeutically active compound is administered.

A “pharmaceutically acceptable salt” refers to a salt of a therapeutically active molecule or compound that is pharmaceutically acceptable and that possesses the desired pharmacological activity of the parent molecule or compound. Pharmaceutically acceptable salts of the therapeutically active agents described herein include those salts derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate salts. Other acids, such as oxalic, while not in themselves pharmaceutically acceptable, may be employed in the preparation of salts useful as intermediates in obtaining pharmaceutically acceptable acid addition salts. Salts derived from appropriate bases include alkali metal (e.g., sodium and potassium), alkaline earth metal (e.g., magnesium), ammonium and salts.

As used herein, “polymer” intends any polymer, copolymer and blends unless otherwise expressly defined. The polymers for use in connection with the compositions and methods of the present disclosure can be produced using standard polymerization and copolymerization techniques, such as graft copolymerization, polycondensation and polyaddition, optionally with an appropriate catalyst. These techniques can be carried out in conventional manner well known in the polymer art as regards to time, temperature and other parameters. Alternatively, the polymers used herein can be produced using standard blending techniques of polymers or blending of copolymers, again carried out in conventional manners well known in the polymer art.

As used herein, the terms “protein” and “polypeptide” are used interchangeably to designate a series of amino acid residues connected to the other by peptide bonds between the alpha-amino and carboxy groups of adjacent residues. The terms thus refer to a polymer of protein amino acids, including modified amino acids (e.g., phosphorylated, glycated, glycosylated, etc.) and amino acid analogs, regardless of size or function. Protein and polypeptide are often used in reference to relatively large polypeptides, whereas the term “peptide” is often used in reference to small polypeptides, but usage of these terms in the art overlaps. The terms “protein” and “polypeptide” are used interchangeably herein when referring to a gene expression product and fragments thereof. Thus, exemplary polypeptides or proteins include gene expression products, naturally occurring proteins, homologs, orthologs, paralogs, fragments and other equivalents, variants, derivatives, fragments, and analogs of the foregoing.

As used herein, a “stroke event” in a subject refers to a medical condition where inadequate blood flow to the brain results in neurological cell death. Stroke events are classified as two main types: ischemic (resulting from an interruption of blood supply to the brain); and hemorrhagic (resulting from rupture of a blood vessel or an abnormal vascular structure). Both types of stroke event result in parts of the brain not functioning properly. An ischemic stroke event is typically caused by blockage of a blood vessel, where blood supply to part of the brain is decreased, leading to dysfunction of the brain tissue in that area. There are four causes for such ischemia; thrombosis; embolism; systemic hypoperfusion; and cerebral venous sinus thrombosis. A hemorrhagic stroke event is caused by either bleeding directly into the brain or into the space between the brain’s membranes, wherein such bleeding may occur due to a ruptured brain aneurysm. There are two main types of hemorrhagic stroke, intracerebral hemorrhage (bleeding within the brain itself); and subarachnoid hemorrhage (bleeding outside the brain tissue but within the brain cavity). Signs and symptoms of a stoke event occurring in a subject are well known in the medical arts and can include sudden-onset face weakness, arm drift, abnormal speech, an inability to move or feel on one side of the body, difficulty in understanding, dizziness or vertigo, loss of vision to one side, and severe headache. An ischemic stroke event can be classified as total anterior circulation infarct (TACI); partial anterior circulation infarct (PACI); lacunar infarct (LACI); or posterior circulation infarct (POCI) that predict the extent of the stroke event, the area of brain affected, the underlying cause and the prognosis. Clinical diagnoses of a stroke event are also well known in the medical arts and are typically based on physical and neurological examination (such as the NIHSS) supported by medical imaging techniques such as CT scan, MRI scan, Doppler ultrasound and arteriography and often supported by ancillary tests such as electrocardiogram (ECG) and blood tests.

The term “subject” as used herein, means any animal (e.g., mammals, including, but not limited to humans, primates, dogs, cattle, cows, horses, kangaroos, pigs, sheep, goats, cats, rabbits, rodents), transgenic non-human animals, fish, amphibians, not limited to frogs, and salamanders, reptiles, other vertebrates and invertebrates and the like, which are to be the recipient of a particular method of treatment.

The terms “systemic” or “systemically” as used herein mean, with respect to delivery or administration of a therapeutically active agent to a subject, that such agent is detectable at a biologically-significant level in the blood plasma of the subject. The term includes oral or parenteral administration of a therapeutically active agent to a subject.

As used herein, the term “therapeutically active” may refer to an activity of a GDF11 molecule or compound whose effect is consistent with a desirable therapeutic outcome in an intended subject. The terms “therapeutically active agent”, “therapeutically active GDF11 molecule” or a “therapeutically active derivative, variant or modified GDF11” are used interchangeably herein and refer to a molecule having a therapeutic activity whose effect is consistent with a desirable outcome in a subject and, in the case of a variant, derivative and/or modified molecule, is consistent with the pharmacological activity of the parent molecule. Therapeutic activity may be measured using in vitro or in vivo methodology well known to those of skill in the relevant art, for example a desirable therapeutic effect can be assayed in cell culture.

A “therapeutically effective amount” refers to the amount of a therapeutically active agent (molecule or compound) that, when administered to a subject, is sufficient to affect a desired treatment for the disease, condition, complication or disorder present in the subject. The “therapeutically effective amount” of a therapeutically active agent for use in any particular method herein will vary depending on the molecule or compound, the disease, condition, complication or disorder, and its severity and the age and weight of the subject. The full therapeutic effect may not necessarily occur by administration of one single dose of the therapeutically active agent (molecule or compound) and may occur only after administration of a series of doses thereof. A therapeutically effective amount may also vary depending on the identity of the active agent(s), the disease, condition, disorder or complication being addressed (and the severity thereof), as well as the age, weight, adsorption, distribution, metabolism and excretion of the relevant active agent in the subject. Thus, a therapeutically effective amount may need to be administered in one or more administrations to the subject. An appropriate therapeutically effective amount of a therapeutically active molecule or compound can be determined according to any one of several well-established protocols known to those of ordinary skill in the relevant art. For example, animal studies, such as studies using mice, rats or larger mammals, can be used to determine an appropriate dose of a pharmaceutical compound. The results from such animal studies can then be extrapolated to determine doses for use in other species, such as for example, humans.

The terms “treating” or “treatment” refer to any amelioration, rehabilitation, rejuvenation, improvement, decrease or mitigation of any one or more affect, complication, decrease in normal or preexisting function or capacity, disability or disorder arising from a stroke event in a subject and/or progression or exacerbation of such affect, complication, decrease in normal or preexisting function or capacity, disability or disorder, or of at least one clinical symptom thereof (e.g., stabilization of a discernible symptom), physiologically (e.g., stabilization of a physical parameter), or both, and/or inhibiting at least one physical parameter which may not be discernible to the subject. “Treating” or “treatment” as used herein also refers to the potential to prohibit a future or further stroke event in the subject. In the practice of the methods of the present disclosure, treating a stroke event entails administration of a therapeutically effective amount of a GDF 11 molecule (both as defined herein) to a subject using a dosing regimen that targets early initiation (within anywhere from about 12 hours to 3 days from the stroke event), high dose administration of the GDF11 molecule (at least 1 mg/kg), and a limited duration of daily treatment (once daily over a period of from 2 to 7 days) can be assessed using diagnostic and clinical examination techniques well known in the art. As discussed herein above, such techniques are typically based on physical and neurological (behavioral) examination (such as the NIHSS) to assess improved body motor function and/or cognitive function in the subject, and can be supported by medical imaging techniques such as CT scan, MRI scan (e.g., spin-echo MRI or cine magnetic resonance), Doppler ultrasound and arteriography and often supported by ancillary tests such as electrocardiogram (ECG) and blood tests. Using such techniques, the ordinarily skilled person can assess successful stroke treatment in a subject by way of visualizing neovascularization, neurogenesis, improved cerebrovascular structure, and/or function or blood flow at or near the site of stroke in a subject. Here again, for clarity, successful stroke therapy using the methods of the present disclosure can be established by assessing any one or more (and any combination thereof) of the above-noted criteria and/or by employing any one or more of the above-noted diagnostic and imaging techniques.

It should be understood that this disclosure is not limited to the particular methodology, protocols, and reagents, etc., described herein and as such can vary. The terminology used herein is for the purpose of describing particular aspects only and is not intended to limit the scope of the present disclosure, which is defined solely by the claims.

### Methods of Treatment, Pharmaceutical Compositions

Methods and compositions are provided for treating stroke in a mammalian subject. The methods employ administration of a therapeutically effective amount of a GDF11 molecule to the subject. The GDF11 molecule is administered to the subject within a limited time window of from about 12 hours to 3 days after a stroke event in the subject, and is carried out under a dosing regimen that features high dose administration of the GDF11 molecule, wherein such dosing is continued for a limited period of from 2 to about 14 days.

It has been discovered, surprisingly, that in a subject in need of treatment after a stroke event, administering a therapeutically effective amount of a GDF11 molecule or a pharmaceutically acceptable salt thereof — when it is administered within a particular time window after the stroke event, at a selected high dose and over a limited period of time — can effectively treat stroke, as described herein. The present disclosure shows that therapeutically effective amounts of the GDF11 molecule for such daily administration and under such a structured dosing regimen preferably includes at least about 0.8 mg/kg-body weight up to about 4 mg/kg- body weight in a rodent subject, or the equivalent (corresponding) amount in a larger subject, of this unique therapeutically active agent.

Thus, it is a primary aspect of the present disclosure to provide a method for treating stroke in a subject. The method entails beginning a dosing regimen by administering a therapeutically effective amount of a GDF11 molecule to the subject within the time frame of about 12 to 72 hours after a stroke event in the subject. The GFD11 molecule is administered in an amount of at least about 0.8 mg per kg body weight of the rodent subject (0.8 mg/kg) or the corresponding amount in a large mammalian subject and is carried out over a period of from 2 to about 14 days. In one aspect of the disclosure, the GDF11 molecule is administered in an amount of at least about 1 mg per kg body weight of the rodent subject (1 mg/kg) or the corresponding amount in a larger subject. In another aspect of the disclosure, the GDF11 molecule is administered for a shortened period of about 7 days, and in yet another aspect, the GDF 11 molecule is administered for an even shorter period of from 2 to 4 days. In one particular aspect of the present disclosure, the method entails administration of a pharmaceutical composition comprising the GDF11 molecule. The composition can further include a pharmaceutically acceptable carrier, excipient or vehicle.

Administration of the GDF11 molecule can be systemic and/or local and is carried out via any suitable parenteral administration technique. For example, the composition can be administered to a subject via standard intravenous, intramuscular, intraperitoneal or subcutaneous injection. In certain aspects, the composition containing the GDF11 molecule can thus include a suitable injection vehicle such as water for injection. Such compositions can further include a controlled release excipient. In the practice of such methods, the composition can be provided in the form of a nanoparticle such as a liposome. These compositions can further include a bioerodible polymer or can be conveniently provided in the form of a solid or injectable implant. In the practice of the compositions and methods of the present disclosure, the GDF11 molecule can be present in the composition in the form of a solution, suspension or emulsion.

In a particular aspect of the present disclosure, the methods are suitable to bring about any amelioration, rehabilitation, rejuvenation, improvement, decrease or mitigation of any one or more affect, complication, decrease in normal or preexisting function or capacity, disability or disorder arising from a stroke event in a subject and/or progression or exacerbation of such affect, complication, decrease in normal or preexisting function or capacity, disability or disorder, or of at least one clinical symptom thereof (e.g., stabilization of a discernible symptom), physiologically (e.g., stabilization of a physical parameter), or both, and/or inhibiting at least one physical parameter.

Successful conduct of the methods of the present disclosure can be assessed using techniques well known in the art. As discussed herein above, successful treatment of a stroke event in a subject can be assessed using diagnostic and clinical examination techniques well known in the art, where such techniques rely upon physical and neurological (behavioral) examination to assess improved body motor function and/or cognitive function in the subject, and can be supported by medical imaging techniques such as CT scan, MRI scan (e.g., spin-echo MRI), Doppler ultrasound and arteriography supplemented by ancillary tests. Using such techniques, the ordinarily skilled person can assess successful stroke treatment in a subject by way of visualizing neovascularization, neurogenesis, improved cerebrovascular structure, and/or function or blood flow at or near the site of stroke in a subject. As discussed herein above, successful stroke therapy using the methods of the present disclosure can be established by assessing any one or more of the above-noted criteria, employing any one or more of the above-noted diagnostic and imaging techniques, or using any other methods of the present disclosure to assess any one or more (and any combination thereof) of the above-noted criteria.

In the practice of the methods of the present disclosure, the selected GDF11 molecule is any therapeutically active form of a GDF11 molecule that can be the same or different in the compositions employed over the course of the dosing regimen(s) disclosed herein. For example, the mature human form of a GDF11 polypeptide is particularly preferred. Alternatively, any derivative, variant or modified form of a “native” GDF11 molecule can be used, so long as such molecule is therapeutically active as defined herein. In some aspects, the GDF11 molecule that is administered to the subject can comprise a GDF11 polypeptide homodimer. The GD11 molecule can also comprise a GDF11 polypeptide or fragment thereof. In some particular aspects of the disclosure, a variant, derivative or fragment of a GDF11 polypeptide is administered to the subject. In this regard, the variant, derivative or fragment of GDF11 can be a conservatively modified variant, derivative or fragment of the native sequence of the mature form of human GDF11. In certain aspects of the disclosure, the subject can be administered a modified GDF11 molecule comprising a second polypeptide moiety selected from Collectin kidney 1 (e.g. NCBI Gene ID No: 78989) (SEQ ID NO: 4), Cathespin D (e.g. NCBI Gene ID No: 1509), Dickkopf-related protein 4 (e.g. NCBI Gene ID No: 27121), Erythrocyte membrane protein 4.1 (e.g. NCBI Gene ID No: 2035), esterase D (e.g. NCBI Gene ID No: 2098), hemoglobin (e.g. NCBI Gene ID No: 3043 or 3047), interleukin-1 receptor accessory protein (e.g. NCBI Gene ID No: 3556), natural killer group 2 member D (e.g. NCBI Gene ID No: 22914), Ras-related C3 botulinum toxin substrate 1 (e.g. NCBI Gene ID No: 5879), GTP-binding nuclear protein Ran (e.g. NCBI Gene ID No: 5901), tissue inhibitor of metalloproteases 3 (e.g. NCBI Gene ID No: 7078), or thymidylate synthase (e.g. NCBI Gene ID No: 7298).

In more particular detail, the GDF11 molecule can be a polypeptide obtained by mutations of native nucleotide sequences. A “variant” or “derivative” as referred to herein, is a polypeptide substantially homologous to a native or reference polypeptide, but which has an amino acid sequence different from that of the native or reference polypeptide because of one or a plurality of deletions, insertions or substitutions. DNA sequences encoding polypeptide molecules encompass sequences that comprise one or more additions, deletions, or substitutions of nucleotides when compared to a native or reference DNA sequence but encode a variant or derivative protein or polypeptide (or fragment thereof) that retains the relevant biological activity relative to the reference protein. One of ordinary skill in the art will recognize that individual substitutions, deletions or additions to a nucleic acid, peptide, polypeptide, or protein sequence which alters a single amino acid or a small percentage, (such as 5% or fewer, or 4% or fewer, or 3% or fewer, or 1% or fewer) of amino acids in the encoded sequence is a “conservatively modified variant” or conservatively modified derivative” where the alteration results in the substitution of an amino acid with a chemically similar amino acid. It is contemplated that some changes can potentially improve the relevant activity, such that a variant or derivative, whether conservative or not, has at least about 90%, more preferably about 95% or 100% of the activity of wildtype GDF11, and even more preferably about 110% or more of the activity of the wildtype (native) form of the relevant GDF11 molecule.

One method of identifying amino acid residues which can be substituted is to align, for example, human GDF11 to a GDF11 homolog from one or more non-human species. Alignment can provide guidance regarding not only residues likely to be necessary for function but also, conversely, those residues likely to tolerate change. Where, for example, an alignment shows two identical or similar amino acids at corresponding positions, it is more likely that that site is important functionally. Where, conversely, alignment shows residues in corresponding positions to differ significantly in size, charge, hydrophobicity, etc., it is more likely that that site can tolerate variation in a functional polypeptide. Similarly, alignment with a related polypeptide from the same species, e.g. GDF8, which does not show the same activity, can also provide guidance with respect to regions or structures required for GDF11 activity. The variant or derivative amino acid sequence can be at least 90%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web. The variant amino acid can be at least 90%, at least 91%, at least 92%, at least 93%, at least 94%, at least 95%, at least 96%, at least 97%, at least 98%, at least 99%, or more, similar to the sequence from which it is derived (referred to herein as an “original” sequence). The degree of similarity (percent similarity) between an original and a mutant sequence can be determined, for example, by using a similarity matrix. Similarity matrices are well known in the art and a number of tools for comparing two sequences using similarity matrices are freely available online, e.g. BLAST (available on the world wide web at http://blast.ncbi.nlm.nih.gov), with default parameters set.

It is noted that the mature GDF11 polypeptide includes likely intrachain disulfide bonds between, e.g., amino acid 313 and 372; 341 and 404; and 345 and 406 (numbered relative to the full length polypeptide, including the signal sequence) and that amino acid 371 likely participates in interchain disulfide bonding.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired apoptotic activity of a native or reference polypeptide is retained. Conservative substitution tables providing functionally similar amino acids are well known in the art. Such conservatively modified variants are in addition to and do not exclude polymorphic variants, interspecies homologs, and alleles consistent with the disclosure. Typical conservative substitutions for one another include: 1) Alanine (A), Glycine (G); 2) Aspartic acid (D), Glutamic acid (E); 3) Asparagine (N), Glutamine (Q); 4) Arginine (R), Lysine (K); and 5) Isoleucine (I), Leucine (L), Methionine (M).

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

The GDF11 polypeptide molecule administered to the subject can also comprise one or more amino acid substitutions, modifications or additions. For example, substitutions and/or modifications or additions can be used to prevent or reduce proteolytic degradation and/or prolong half-life of the GDF11 molecule in the subject. The GDF11 polypeptide can also be modified by conjugating or fusing it to other polypeptide or polypeptide domains such as, by way of non-limiting example, transferrin, albumin, growth hormone; cellulose and/or Fc fragments (see, e.g., U.S. Pat. No. 9,434,779). The GDF11 polypeptide can also be modified by conjugation or fusion to the growth and differentiation factor 8 (GDF8) precursor moiety, or any fragment or derivative thereof. Alternatively or additionally, the GDF11 polypeptide as described herein can comprise at least one peptide bond replacement. A single peptide bond or multiple peptide bonds, e.g. 2 bonds, 3 bonds, 4 bonds, 5 bonds, or 6 or more bonds, or all the peptide bonds can be replaced. In other aspects, a GDF 11 polypeptide molecule as described herein can comprise one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

In yet other aspects of the disclosure, a GDF11 polypeptide molecule for use in the current methods can be comprised of naturally occurring amino acids commonly found in polypeptides and/or proteins produced by living organisms, e.g., Ala (A), Val (V), Leu (L), Ile (I), Pro (P), Phe (F), Trp (W), Met (M), Gly (G), Ser (S), Thr (T), Cys (C), Tyr (Y), Asn (N), Gln (O), Asp (D), Glu (E), Lys (K), Arg (R), and His (H). In other aspects, the GDF11 polypeptide molecule can include alternative amino acids. Non-limiting examples of alternative amino acids include, D-amino acids; beta-amino acids; homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

In still further aspects of the disclosure, a modified GDF11 polypeptide molecule can be selected, e.g., such as a molecule modified by addition of a moiety to one or more of the amino acids comprising the polypeptide. For example, a GDF11 polypeptide as described herein can comprise one or more moiety molecules, e.g., 1 or more moiety molecules per peptide, 2 or more moiety molecules per peptide, 5 or more moiety molecules per peptide, 10 or more moiety molecules per peptide or more moiety molecules per peptide. Suitable GDF11 modified polypeptides can include one more types of modifications and/or moieties, e.g., 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; albumin, and cyclization. Alternatively, an end-capping modification can comprise acetylation at the N-terminus, N-terminal acylation, and N-terminal formylation, or an end-capping modification can comprise amidation at the C-terminus, introduction of C-terminal alcohol, aldehyde, ester, and thioester moieties. The half-life of a modified GDF11 polypeptide can thus be increased by the addition of selected such moieties, e.g., PEG or albumin. In any event, the GDF11 molecule can be modified through known medical chemistry techniques to improve at least one of bio-distribution, ease of administration, metabolic stability, and a combination of at least two thereof.

In other aspects of the disclosure, a modified GDF11 polypeptide molecule can be presented as a pharmaceutically acceptable prodrug. As used herein, a “prodrug” refers to a compound that can be converted via some chemical or physiological process (e.g., enzymatic processes and metabolic hydrolysis) to a therapeutically active agent. Thus, the term also refers to a precursor of a therapeutically active compound that is pharmaceutically acceptable. A prodrug may be inactive when administered to a subject, e.g., as an ester, but is converted in vivo to an active compound, for example, by hydrolysis to the free carboxylic acid or free hydroxyl. The prodrug molecule often offers advantages of solubility, tissue compatibility or delayed release in a subject. A prodrug can also include any covalently bonded carriers, which release the active compound in vivo when such prodrug is administered to a subject. Prodrugs of an active compound can be prepared by modifying functional groups present in the active molecule in such a way that the modifications are cleaved, either in routine manipulation or in vivo, to the parent active molecule. Prodrugs include molecules wherein a hydroxy, amino or mercapto group is bonded to any group that, when the prodrug is administered to a subject, it cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. See, e.g., Harper, Drug Latentiation in Jucker, ed. Progress in Drug Research 4:221-294 (1962); Morozowich et al, Application of Physical Organic Principles to Prodrug Design in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. (1999) Curr. Pharm. Design. 5(4): 265-287; Pauletti et al. (1997) Adv. Drug. Delivery Rev. 27: 235-256; Mizen et al. (1998) Pharm. Biotech. 11: 345-365; Gaignault et al. (1996) Pract. Med. Chem. 671-696; Asgharnejad, Improving Oral Drug Transport, in Transport Processes in Pharmaceutical Systems, G. L. Amidon, P. I. Lee and E. M. Topp, Eds., Marcell Dekker, p. 185-218 (2000); Balant et al. (1990) Eur. J. Drug Metab. Pharmacokinet., 15(2): 143-53; Balimane et al. (1999) Adv. Drug Delivery Rev. 39(1-3): 183-209; Browne (1997) Clin. Neuropharmacol. 20(1): 1-12; Bundgaard H. (1979) Arch. Pharm. Chem 86(1): 1-39; Bundgaard H. (1987) Controlled Drug Delivery 17: 179-96; Bundgaard H. (1992) Arfv. Drug Delivery Rev. 8(1): 1-38; Fleisher et al. (1996) Arfv. Drug Delivery Rev. 19(2): 115-130; Fleisher et al. (1985) Methods Enzymol. 112: 360-81; Farquhar D, et al. (1983) Pharm. Sci. 72(3): 324-325; Freeman et al. (1991) Chem. Soc., Chem. Commun. 875-877; Friis et al. (1996) Eur. J. Pharm. Sci. 4: 49-59; Gangwar et al. (1977) Des. Biopharm. Prop. Prodrugs Analogs, [Symp.] Meeting Date 1976: 409-21; Nathwani et al. (1993) Drugs 45(6): 866-94; Sinhababu et al. (1996) Adv. Drug Delivery Rev. 19(2): 241-273; Stella et al. (1985) Drugs 29(5): 455-73; Tan et al. (1999) Adv. Drug Delivery Rev. 39(1-3): 117-151; Taylor (1996) Adv. Drug Delivery Rev. 19(2): 131-148; Valentino et al. (1997) Drug Discovery Today 2(4): 148-155; Wiebe et al. (1999) Adv. Drug Delivery Rev. 39(1-3): 63-80 (1999); and Waller et al. (1989) Br. J. Clin. Pharmac. 28: 497-507.

Suitable GDF11 polypeptide molecules for use herein can be synthesized by using well known methods including recombinant methods and chemical synthesis. Recombinant methods of producing a peptide through the introduction of a vector including nucleic acid encoding the peptide into a suitable host cell are well known in the art, such as those described in Sambrook et al., Molecular Cloning: A Laboratory Manual, 2d Ed, Vols 1 to 8, Cold Spring Harbor, N.Y. (1989); M. W. Pennington and B. M. Dunn, Methods in Molecular Biology Peptide Synthesis Protocols, Vol 35, Humana Press, Totawa, N.J. (1994). Suitable polypeptides can also be chemically synthesized using methods well known in the art (see, e.g., Merrifield et al. (1964) J. Am. Chem. Soc. 85: 2149; Bodanszky, M. (1984) Principles of Peptide Synthesis, Springer-Verlag, New York, N.Y.; Kimmerlin et al. (2005) Pept. Res. 65: 229-260; Nilsson et al. (2005) Annu. Rev. Biophys. Biomol. Struct. 34: 91-118; W. C. Chan and P. D. White (Eds.) Fmoc Solid Phase Peptide Synthesis: A Practical Approach, Oxford University Press, Cary, N.C. (2000); N. L. Benoiton, Chemistry of Peptide Synthesis, CRC Press, Boca Raton, Fla. (2005); J. Jones, Amino Acid and Peptide Synthesis, 2.sup.nd Ed, Oxford University Press, Cary, N.C. (2002); and P. Lloyd-Williams, F. Albericio, and E. Giralt, Chemical Approaches to the Synthesis of Peptides and Proteins, CRC Press, Boca Raton, Fla. (1997). Peptide derivatives can also be prepared as described in U.S. Pat. Nos. 4,612,302; 4,853,371 and 4,684,620; and in U.S. Pat. App. Pub. No. 2009/0263843.

Alterations of the original amino acid sequence of a GDF11 polypeptide molecule can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed in U.S. Pat. No. 9,434,779, incorporated herein by reference in its entirety. In some aspects of the instant disclosure, a modified, variant or derivative GDF11 polypeptide molecule can be chemically synthesized and mutations can be incorporated as part of the chemical synthesis process.

The selected GDF11 molecule is then formulated as a pharmacological composition for use in administration to the subject. For example, the GDF11 molecule can be provided as a pharmaceutically acceptable solvate. The term “solvate” refers to a GDF11 molecule as described herein in the solid state, wherein molecules of a suitable solvent are incorporated in the crystal lattice. A suitable solvent for therapeutic administration is physiologically acceptable at the dosage administered. Examples of suitable solvents for therapeutic administration are ethanol and water. When water is the solvent, the solvate is referred to as a hydrate. In general, solvates are formed by dissolving the molecule in the appropriate solvent and isolating the solvate by cooling or using an antisolvent. The solvate is typically dried or azeotroped under ambient conditions.

Proper preparation of a pharmaceutical composition that contains an active ingredient (the GDF11 molecule) dissolved or dispersed therein is well understood in the art and generally need not be limited based on formulation. Typically, such compositions are prepared as an injectable either as liquid solutions or suspensions, however, solid forms suitable for solution, or suspension, in liquid prior to use can also be prepared. The preparation can also be emulsified or presented as a liposome composition. The GDF11 molecule can be mixed with excipients which are pharmaceutically acceptable and compatible with the GDF11 molecule and in amounts suitable for use in the methods described in this disclosure. Suitable excipients are, for example, water, saline, dextrose, glycerol, ethanol or the like and combinations thereof. In addition, if desired, a pharmaceutical composition can contain minor amounts of auxiliary substances such as wetting or emulsifying agents, pH buffering agents, and the like which enhance the effectiveness of the active ingredient. The composition of the present disclosure can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic, and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine, and the like. Pharmaceutically acceptable carriers, excipients and vehicles are well known in the art. Exemplary liquid carriers are sterile aqueous solutions that contain no materials in addition to the active ingredients and water, and/or can contain a buffer such as sodium phosphate at physiological pH value, physiological saline or both, such as phosphate-buffered saline. In still further aspects of the disclosure, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of the GDF11 molecule used in the present methods that will be effective in the treatment of a stroke disorder or condition in a subject will depend on the nature of such disorder or condition and can be determined by standard clinical techniques.

In certain aspects of the present disclosure, the GDF11 molecule can be administered using a controlled release dosage form or composition. Controlled release pharmaceutical products have a common goal of improving drug therapy over that achieved by their non-controlled release counterparts. Ideally, the use of an optimally designed controlled release dosage form or composition in medical treatment is characterized by a minimum of drug substance being employed to address the disorder or condition in a minimum amount of time. Advantages of controlled release approaches include: 1) extended activity of the GDF11 molecule; 2) reduced dosage frequency; 3) increased compliance; 4) potential for use of less total GDF11 in the dosage form; 5) reduction in local or systemic side effects; 6) minimization of drug (GDF11) accumulation; 7) reduction in blood level fluctuations; 8) improvement in efficacy of treatment; 9) reduction of potentiation or loss of drug (GDF11) activity; and/or 10) improvement in speed of control of diseases or conditions.

Conventional dosage forms and compositions generally provide rapid or immediate drug release from the formulation. Depending on the pharmacology and pharmacokinetics of the drug, use of conventional dosage forms can lead to wide fluctuations in the concentrations of the drug in a subject’s blood and other tissues. These fluctuations can impact a number of parameters, such as dose frequency, onset of action, duration of efficacy, maintenance of therapeutic blood levels, toxicity, side effects, and the like. Advantageously, controlled release dosage forms or compositions can be used to control the GDFD11 molecule’s onset of action, duration of action, plasma levels within the therapeutic window, and peak blood levels. In particular, controlled release can be used to ensure that the maximum effectiveness of the GDF11 molecule is achieved while minimizing potential adverse effects and safety concerns, which can occur both from under-dosing a drug (i.e., going below the minimum therapeutic levels) as well as exceeding the toxicity level for the drug.

Most controlled-release dosage forms or compositions are designed to initially release an amount of drug (active ingredient) that promptly produces the desired therapeutic effect, and gradually and continually release further amounts of drug to maintain this level of pharmacological effect over an extended period of time. In order to maintain a constant level of drug in the body, the drug must be released from the dosage form at a rate that will replace the amount of drug being metabolized and excreted from the body. Controlled release of an GDF11 molecule from a selected dosage form or composition can be stimulated by various conditions including, but not limited to, pH, ionic strength, osmotic pressure, temperature, enzymes, water, and other physiological conditions, molecules or compounds. A variety of known controlled release dosage forms and compositions can be adapted for use in the methods of this disclosure. Examples include, but are not limited to, those described in U.S. Pat. Nos. 3,845,770; 3,916,899; 3,536,809; 3,598,123; 4,008,719; 5674,533; 5,059,595; 5,591,767; 5,120,548; 5,073,543; 5,639,476; 5,354,556; 5,733,566; and 6,365,185. These dosage forms provide for controlled release using excipients such as hydroxypropylmethyl cellulose, other polymer matrices, gels, permeable membranes, or osmotic systems, or a combination thereof to provide the desired release profile.

The GDF11 molecule is included in the composition in an amount sufficient to exert a therapeutically useful effect in the absence or minimization of undesirable side effects in the subject. Such therapeutically effective concentration may be predicted empirically by testing the GDF11 molecule in in vitro and in vivo systems well known to those of skill in the art and then extrapolated therefrom for dosages for humans. Human doses are then typically fine-tuned in clinical trials and titrated to bring about the desired therapeutic response. To formulate a composition, the weight fraction of a compound is dissolved, suspended, dispersed or otherwise mixed in a selected carrier, excipient or vehicle at an effective concentration. The formulated pharmaceutical compositions containing the GDF11 molecule can then be conventionally administered in the form of a unit dose, for example. The term “unit dose” when used in reference to a pharmaceutical composition refers to physically discrete units suitable as unitary dosage for the subject, each unit containing a predetermined quantity of the GDF11 molecule calculated to produce the desired pharmacological effect in association with a pharmaceutically acceptable carrier, excipient or vehicle. Examples of unit dose forms include ampoules and syringes. Thus, in one preferred aspect of the disclosure, the GDF11 molecule is provided in the form of a pharmaceutical composition that includes water for injection. In related aspects, a syringe comprising a therapeutically effective amount of the GDF11 molecule in a pharmaceutical composition is provided. Unit-dose forms may be administered in fractions or multiples thereof. A multiple dose form is a plurality of identical unit dose forms packaged in a single container to be administered in segregated unit dose form. Examples of multiple dose forms include vials, bottles of tablets or capsules or bottles of pints or gallons. Hence, a multiple dose form is a multiple of unit doses which are not segregated in packaging. Alternatively, the GDF11 composition is provided in a kit (e.g., a package or container) including at least one therapeutically active agent (a GDF11 molecule). In certain kits the manufacture may be labeled, promoted, distributed, or sold as a unit for performing the methods of the present disclosure.

As discussed herein above, preferred routes of administration for the present compositions are parenteral, e.g., via intravenous, intramuscular, intraperitoneal, intradermal or subcutaneous injection. Solutions or suspensions used for such parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH of a composition can be adjusted with acids or bases, such as hydrochloric acid or sodium hydroxide. Parenteral preparations can be enclosed in ampoules, disposable syringes or multiple dose vials made of glass or plastic. Pharmaceutical compositions suitable for injection include sterile aqueous solutions (where water soluble) or dispersions, emulsions or suspensions and sterile powders for the extemporaneous preparation of sterile injectable solutions or dispersions. For intravenous administration, suitable carriers comprise physiological saline, bacteriostatic water, Cremophor EL.TM. (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier or vehicle can be a solvent or dispersion medium containing, for example, water, ethanol, polyol (for example, glycerol, propylene glycol, and liquid polyetheylene glycol, and the like), and suitable mixtures thereof. The proper fluidity of a composition can be maintained, for example, by the use of a coating such as lecithin, by the maintenance of the selected particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some cases, isotonic agents are included in the composition, for example, sugars, polyalcohols such as manitol, sorbitol, or sodium chloride. Prolonged absorption of an injectable composition can be achieved by including in the composition an excipient that delays absorption, for example, aluminum monostearate or gelatin.

Sterile injectable solutions can be prepared by incorporating the GDF11 molecule in a specified amount in an appropriate solvent with one or a combination of ingredients enumerated above, as needed, followed by filtered sterilization. Generally, dispersions are prepared by incorporating the GDF11 molecule into a sterile vehicle that contains a basic dispersion medium and other ingredients selected from those enumerated above or others known in the art. In the case of sterile powders for the preparation of sterile injectable solutions, the methods of preparation include vacuum drying and freeze-drying which yields a powder of the GDF11 molecule plus any additional desired ingredient from a previously sterile-filtered solution thereof.

In certain aspects of the present disclosure, the instant compositions are suitable for implantation in the subject. For example, implantable devices or systems can be configured as a shaped article, such as a sphere, rod, slab, film, fiber, needle, cylinder, sheet, tube, or any other suitable geometry including microparticles, microspheres, and/or microcapsules. The implants can be provided any suitable size and shape for specialized locations, for example as a catheter, shunt, device for continuous subarachnoid infusion, feeding tube, solid implant to prevent surgical adhesion, uterine implant, artificial sphincter, periurethral implant, splint, opthlamic implant, contact lens, plastic surgery implant, stent (containing or coated with the active agent) including an esophageal stent, gastrointestinal stent, vascular stent, biliary stent, colonic stent, pancreatic stent, ureteric stent, urethral stent, lacrimal stent, Eustachian tube stent, fallopian stent, nasal stent, sinus stent, tracheal stent, or bronchial stent, or a port including a venous access device, implanted port, epidural catheter or central catheter (PICC). The implants can be implanted at a desired site surgically, or using minimally invasive techniques employing trocars, catherers, etc. The implants can alternatively be implanted into any suitable tissue using standard techniques, such as implanted intradermally, subdermally, subcutaneously, intraperitoneally, intramuscularly, or intralumenally (e.g., intraarterially, intravenously, intravaginally, rectally, or into the periodontal space). The implants can alternatively be fabricated as part of a matrix, graft, prosthetic or coating. If an implantable device is manufactured in particulate form, e.g., as a microparticle, microsphere or microcapsule, it can then be implanted into suitable tissue using a cannula, needle and syringe or like instrument to inject a suspension of the particles.

Administration of the GDF11 molecule is typically carried out intravenously by way of a catheter such as a central venous catheter line or like IV catheter. Alternatively, the GDF11 composition can be administered via intravenous, intramuscular, intraperitoneal or subcutaneous injection using a standard needle and syringe. In certain aspects, the composition can thus be simply formulated to include a suitable injection vehicle such as water for injection. In yet other aspects, the composition can be administered using an external drug pump such as an infusion pump. In the practice of the methods of the present disclosure, the GDF11 molecule can be present in the composition in the form of a solution, suspension or emulsion.

In a particular aspect of the present disclosure, the precise dosage and duration of treatment employed in the practice of the methods is a function of the type of stroke and resulting stroke damage that is being addressed and can be determined empirically using known testing protocols or by extrapolation from in vivo or in vitro test data or subsequent clinical testing. It is to be noted that concentrations and dosage values can also vary with the severity of the stroke to be addressed. For any particular subject, specific dosage regimens can be adjusted over time according to the individual need and the professional judgment of the person administering or supervising the administration of the present GDF11 compositions and the concentration ranges set forth herein are exemplary only and are not intended to limit the scope or practice of the claimed methods.

In other aspects of the disclosure, administration of the compositions can be carried on any suitable basis, such as on a once daily (QD) basis, twice daily (BID), three times daily (TID), four times daily (QID), hourly (“q_h” where “h” denotes the number of hours between doses), or the like, and each day of treatment can be the same or different over the course of treatment. In other aspects of the disclosure, treatment can be carried out once, or can entail any number of treatment regimens suitable for the specific treatment being contemplated. For example, a suitable treating regimen can include a first administration (on day one of the treatment) of the GDF11 molecule at a first dose followed by a second administration or subsequent administrations of the GDF 11 molecule at a second or more higher or lower dose (e.g., on day 2 up to day 14 of the treatment). In some variations, the GDF11 treatment regimen can be carried out for a one day period. In some variations, the GDF11 treatment regimen can be carried out for a two day period. In some variations, the GDF11 treatment regimen can be carried out for a three day period. In some variations, the GDF11 treatment regimen can be carried out for a four day period. In some variations, the GDF11 treatment regimen can be carried out for a five day period. In some variations, the GDF11 treatment regimen can be carried out for a six day period. In some variations, the GDF11 treatment regimen can be carried out for a seven day period. In some variations, the GDF11 treatment regimen may be carried out on intermittent days. In such administration, any one, two, or three days can be skipped in any combination. For example, dosing can be carried out on Day 1, Day 3, and Day 6; Day 1, Day 3, and Day 7, Day 1, Day 2, Day 4, and Day6; Day 1, Day 1, Day 2, Day 3, Day 5, and Day 7; and the like.

In certain aspects, the dosing regimen entails classical titration of the GDF11 molecule in either ascending or descending doses, for example wherein the first administration is carried out at an initial dose of at least the minimal high dose of GDF11 on day 1 of the treatment period and finishes at a second, higher dose, with any number of different intervening doses carried out between such first and second doses. Alternatively, titration of the GDF11 molecule can entail an initial (day one) high dose of the GDF11 molecule and ending with a final dose of at least the minimal high dose of GDF11, again with any number of different intervening doses carried out between such initial and final doses. In any titration strategy, it may be preferred to administer the GDF11 molecule at a first high dose approaching the median toxic dose (MTD) for that molecule, or at least approaching the maximum dose of the therapeutic window for the administered GDF11 molecule, followed by a subsequent dose (or doses) at lower level.

In other aspects of the disclosure, the GDF11 treatment regimens can be carried out multiple times (e.g., repeated), with a so-called “drug holiday”, that is, by following a structured treatment interruption, tolerance break or treatment break, e.g., where subsequent treatment(s) occur from 2 to 7 days after completion of the initial treatment. Here again, for any particular subject, specific dosing regimens can be adjusted over time according to the individual need and the professional judgment of the person administering or supervising the administration of the present GDF 11 compositions and the dosing strategies set forth herein are exemplary only and are not intended to limit the scope or practice of the claimed methods. In one particular regimen, the first or first few administrations of the GDF11 molecule are carried out in an intensive care setting where the subject has a catheter such as a central venous catheter line or like IV catheter. Upon stabilization and move to a step-down unit, stroke recovery unit, or other suitable setting, subsequent administrations of the GDF11 molecule can then be carried out using a needle and syringe. Subsequent treatment regimens (for example after a drug holiday) can be carried out using an implant or an external drug pump. Subsequent treatment regimens can target the same high dose, short duration of administration period as the initial treatment, or can target lower dose administration of GDF11 molecule with or without an extended duration of treatment.

In yet other aspects, the above methods are practiced wherein a therapeutically effective amount of the GDF11 molecule is administered in combination with at least one additional active agent to achieve an additive or synergistic effect. In certain preferred aspects of the present disclosure, methods are provided wherein the GDF11 molecule and the second therapeutically active agent can be administered concomitantly, as an admixture, separately and simultaneously, separately and concurrently, or separately and sequentially. For example, the GDF11 molecule can be administered with the additional active agent according to one of the group of administration methods including: i) administering simultaneously but separately at least one dose of the additional active agent and at least one dose of the GDF11 molecule; ii) administering together in an admixture at least one dose of the additional active agent and at least one dose of the GDF11 molecule; iii) administering sequentially at least one dose of the additional active agent and at least one dose of the GDF11 molecule, the at least one dose of the additional active agent being administered prior to administration of the at least one dose of the GDF11 molecule; iv) administering sequentially at least one dose of the additional active agent and at least one dose of the GDF11 molecule the at least one dose of the additional active agent being administered following administration of the at least one dose of the GDF11 molecule; and v) administering sequentially and together in an admixture at least one dose of the additional active agent and at least one dose of the GDF11 molecule. Such administration strategies can be predicted empirically by testing the various combinations and sequences in in vitro and in vivo systems well known to those of skill in the art and then extrapolated therefrom for use in human subjects. Human doses are then typically fine-tuned in clinical trials and titrated to response.

Accordingly, a summary of aspects of the claimed invention as supported by the above description and the following examples is as follows: (1) a method for treating stroke in a subject, comprising administration of a therapeutically effective amount of a growth differentiation factor 11 (GDF11) molecule to the subject within 12 to 72 hours after a stroke event in the subject, wherein the GDF11 molecule is administered in an amount of at least the minimal high dose of GDF11 relative to the body weight of the subject per day over a treatment period of from 1 to about 14 days;
(2) the method of (1) wherein administration of the GDF11 molecule is initiated within 1to 24 hours after the stroke event;
(3) the method of any one of (1) or (2) wherein administration of the GDF11 molecule is carried out over a treatment period of from 2 to 4 days;
(4) the method of any one of (1) to (3) wherein the GDF11 molecule is administered to the subject on a once daily (QD) basis, or alternatively once daily on intermittent days;
(5) the method of any one of (1) to (4) wherein the GDF11 molecule is a mature form of a GDF11 polypeptide;
(6) the method of (5) wherein the mature form of the GDF11 polypeptide forms a homodimer;
(7) the method of any one of (1) to (6) wherein the GDF11 molecule is a polypeptide with at least 91% sequence homology with the native sequence of the human GDF11 molecule;
(8) the method of (7) wherein the GDF11 molecule is recombinant human GDF11 (rhGDF11);
(9) the method of (7) wherein the GDF11 molecule is a therapeutically active variant of the human GDF11 molecule;
(10) the method of (7) wherein the GDF11 molecule is a therapeutically active derivative of the human GDF11 molecule;
(11) the method of any one of (9) or (10) wherein the variant or derivative GDFmolecule comprises one or more amino acid substitutions or deletions relative to the native sequence of the human GDFmolecule;
(12) the method of (11) wherein the GDF11 molecule comprises an amino acid analog;
(13) the method of (7) wherein the GDF11 molecule is a modified GDF11 polypeptide;
(14) the method of (13) wherein the modified GDF11 polypeptide is phosphorylated, glycated, glycosylated, pegylated, HESylated, ELPylated, lipidated, acetylated, amidated, end-capped, includes a cyano group, albumin, or is cyclized;
(15) the method of (13) wherein the modified GDF11 polypeptide is a chimeric polypeptide comprising a first GDF11 molecule moiety and a second moiety;
(16) the method of (15) wherein the second moiety is derived from transferrin, growth hormone or an Fc fragment;
(17) the method of any one of (13) to (16) wherein the modified GDF11 polypeptide has an increased half-life relative to a native GDF11 polypeptide;
(18) the method of any one of (1) to (17) wherein the GDF11 molecule is administered to the subject in the form of a pharmaceutical composition comprising a pharmaceutically acceptable carrier, excipient or vehicle;
(19) the method of (18) wherein the pharmaceutical composition comprises water for injection;
(20) the method of (19) wherein the pharmaceutical composition is administered to the subject via intravenous injection;
(21) the method of any one of (1) to (20) wherein dosing of the GDF11 molecule is titrated upwards from an initial minimal high dose on day 1 of the administration period to a higher dose on the final day of the administration period;
(22) the method of any one of (1) to (20) wherein dosing of the GDF11 molecule is titrated downwards from an initial high dose to a final minimal high dose on the final day of the administration period;
(23) the method of any one of (1) to (22) further comprising a second administration of a therapeutically effective amount of a GDF11 molecule to the subject, wherein the second administration is carried out after from 2 to 7 days after completion of the initial administration;
(24) the method of any one of (1) to (23) wherein treatment of the subject is characterized by improved body motor or cognitive function in the subject;
(25) the method of any one of (1) to (24) wherein the stroke event is ischemic;
(26) the method of any one of (1) to (24) wherein the stroke event is hemorrhagic;
(27) the method of any one of (1) to (26) wherein treatment of the subject is characterized by neovascularization, improved cerebrovascular structure, function or blood flow at or near the site of the stroke in the subject;
(28) a composition comprising a therapeutically effective amount of a growth differentiation factor 11 (GDF11) molecule for use in a method for treating stroke in a subject, said method comprising initiating administration of the composition to the subject within 12 to 72 hours after a stroke event in the subject, wherein the composition comprises the GDF11 molecule in an amount of at least the minimal high dose of GDF11 relative to the body weight of the subject per day and the composition is administered to the subject over a treatment period of from 1 to about 14 days;
(29) the composition of (28) wherein the GDF11 molecule is a mature form of a GDF11 molecule;
(30) the composition of (29) wherein the mature form of the GDF11 polypeptide forms a homodimer;
(31) the composition of any one of (28) to (30) wherein the GDF11 molecule is a polypeptide with at least 91% sequence homology with the native sequence of the human GDF11 molecule;
(32) the composition of (31) wherein the GDF11 molecule is recombinant human GDF11 (rhGDF11);
(33) the composition of (31) wherein the GDF11 molecule is a therapeutically active variant of the human GDF11 molecule;
(34) the composition of (31) wherein the GDF11 molecule is a therapeutically active derivative of the human GDF11 molecule;
(35) the composition of any one of (33) or (34) wherein the variant or derivative GDF11 molecule comprises one or more amino acid substitutions or deletions relative to the native sequence of the human GDF11 molecule;
(36) the composition of (31) wherein the GDF11 molecule comprises an amino acid analog;
(37) the composition of (31) wherein the GDF11 molecule is a modified GDF11 polypeptide;
(38) the composition of (37) wherein the modified GDF11 polypeptide is phosphorylated, glycated, glycosylated, pegylated, HESylated, ELPylated, lipidated, acetylated, amidated, end-capped, includes a cyano group, albumin, or is cyclized;
(39) the composition of (37) wherein the modified GDF11 polypeptide is a chimeric polypeptide comprising a first GDF11 molecule moiety and a second moiety;
(40) the composition of (39) wherein the second moiety is derived from transferrin, growth hormone or an Fc fragment;
(41) the composition of any one of (37) to (40) wherein the modified GDF11 polypeptide has an increased half-life relative to a native GDF11 polypeptide;
(42) the composition of any one of (28) to (41) further comprising a pharmaceutically acceptable carrier, excipient or vehicle;
(43) the composition of (42) wherein the pharmaceutical composition comprises water for injection;
(44) the composition of any one of (42) or (43) wherein the composition is formulated for administration to the subject via intravenous injection;
(45) the composition of any one of (28) to (44) for use in a method for treating ischemic stroke in the subject; and
(46) the composition of any one of (28) to (44) for use in a method for treating hemorrhagic stroke in the subject.

It is to be understood that the above description is intended to be illustrative and not restrictive. Many other aspects will be apparent to those of skill in the art upon reading the above description. It should be noted that specific aspects discussed in different portions of the description and/or referred to in the figures can be combined to form additional aspects of the present disclosure. The scope of the disclosure should, therefore, be determined with reference to the appended claims, along with the full scope of equivalents to which such claims are entitled. All publications, patents and patent documents are incorporated by reference herein, as though individually set forth herein in their entirety.

### EXAMPLES

The disclosure is illustrated herein by the experiments described by the following examples, which should not be construed as limiting. Those skilled in the art will understand that this disclosure may be embodied in many different forms and should not be construed as limited to the aspects set forth herein. Rather, these examples are provided so that this disclosure will fully convey the disclosure to those skilled in the art. Many modifications and other aspects of the disclosure will come to mind in one skilled in the art to which this disclosure pertains having the benefit of the teachings presented in the foregoing description. Although specific terms are employed, they are used as in the art unless otherwise indicated.

### Example 1

The purpose of this example was to evaluate the therapeutic effect of a GDF11 molecule administration regimen consisting of a single dose of rGDF 11 administered once daily (QD) for 14 days post-occlusion in a rat permanent Middle Cerebral Artery Occlusion Model (pMCAO) study where body motor function was measured out to 28 days post occlusion. The pMCAO methodology and model is a validated rodent model of stroke recovery that has been used to support initiation of human clinical trials (see, e.g., Iaci et al. (2013) Stroke 44: 1942-1950 (dalfampridine) and Iaci et al. (2016) Journal of Neuroscience Research 94: 253-265 (Neuregulin 1ß3, Glial Growth Factor)).

The pMCAO Model. Focal cerebral infarcts were made by permanent occlusion of the proximal right middle cerebral artery (MCA) using a modification of the method of Tamura and colleagues (see Tamura et al. (1986) No To Shinkei 38: 747-751; Iaci et al. (2013) Stroke 44(7): 1942-1950; and Iaci et al. (2016) J Neurosci Res 94(3): 253-265). Male Sprague-Dawley rats (300-400 g at the time of surgery) were anesthetized with 3% isoflurane in the mixture of N₂O:O₂ (2:1) and were maintained with 2-2.5% isoflurane in the mixture of N₂O:O₂ (2:1). The temporalis muscle was bisected and reflected through an incision made midway between the eye and the eardrum canal. The proximal MCA was exposed through a subtemporal craniectomy without removing the zygomatic arch and without transecting the facial nerve. The artery was then occluded by microbipolar coagulation from just proximal to the olfactory tract to the inferior cerebral vein. Using a rectal temperature probe and connected heating pad, targeted body temperature was set at 37.0 ± 1° C. throughout the procedure. Cefazolin (40 mg/kg; Hospira, Lot: 319002.1, Exp: 28Feb22) was given by intraperitoneal injection (i.p.) before surgery to prevent infections. Buprenorphine, administered subcutaneously (s.c), (~0.1 mg/kg, Simbadol, Lot: B195093, Exp: 31Oct2020) was also given before the MCAO surgery as analgesia.

Animals and Animal Preparation. This study included 2 experimental groups with 12 animals per group totaling 24. All rats were housed and handled for behavioral assessment for five (5) days prior to surgery for acclimation purposes. At the end of the handling period, rats were randomized and assigned to different groups per cage. Twenty-four (24), adult, male Sprague-Dawley rats as described above were used for the study. Rats were randomized and assigned to different groups by cage. Rats were given a unique identification number by tail marking.

Dosing. Animals were dosed starting on day 3 post the surgical occlusion of the MCA with dosing continuing daily, through day 16 after the MCAO. Animals received vehicle at 1 ml/kg or rGDF11 i.p. 1 mg/kg (1 ml/kg) once daily.

Body Weight. Animals were weighed daily from day of surgery until the last day of dosing (Day 16) then Day 21 and Day 28 post MCAO.

Blood Collection. Blood samples (about 300 micro liters (µL) whole blood) were collected an hour after the last doing on Day 16 and was processed for serum. Serum samples will be assessed for mechanistic biomarkers of rGDF11 activity in vivo.

Functional Behavioral Tests. Functional activities were evaluated using limb placing and body swing behavioral tests. Behavioral tests were done before drug administration on days when both were done.

Limb Placing. Limb placing tests were divided into both forelimb and hindlimb tests. The forelimb placing test scored the rat’s ability to place its forelimb on a tabletop in response to whisker, visual, tactile or proprioceptive stimulation. The hindlimb placing test scored the rat’s ability to place its hindlimb on the tabletop in response to tactile and proprioceptive stimulation. Together, these tests reflect function and recovery in the sensorimotor systems (see, e.g., De Ryck et al. (1992) Brain Res 573:44-60). For the forelimb-placing test, the examiner held the rat close to a tabletop and scored the rat’s ability to place the forelimb on the tabletop in response to whisker, visual, tactile, or proprioceptive stimulation. Similarly, for the hindlimb placing test, the examiner assessed the rat’s ability to place the hindlimb on the tabletop in response to tactile and proprioceptive stimulation. Separate sub-scores were obtained for each mode of sensory input (half-point designations possible) and added to give total scores (for the forelimb placing test: 0 = normal, 12 = maximally impaired; for the hindlimb placing test: 0 = normal; 6 = maximally impaired). These tests were performed one day before surgery (Day -1 or Day-pre), one day after surgery (Day 1), three (Day 3), seven (Day 7), fourteen (Day 14) days, twenty-one (Day 21) days and twenty-eight (Day 28) days after MCAO. (Day 0 = day of MCAO).

Body Swing Test. The rat was held approximately one inch from the base of its tail. It was then elevated to an inch above a surface of a table. The rat was held in the vertical axis, defined as no more than 10° to either the left or the right side. A swing was recorded whenever the rat moved its head out of the vertical axis to either side. The rat must have returned to the vertical position for the next swing to be counted. Thirty (30) total swings were counted. The body swing test reflects symmetry of striatal function (see, e.g., Borlongan et al. (1995) J Neurosci 15: 5372-5378). A normal rat typically has an equal number of swings to either side. Following focal ischemia, the rat tends to swing to the contralateral (left) side. The test was performed at the same time of the Limb Placing test. This test reflects symmetry of striatal function (ref) and a normal rat typically has an equal number of swings to either side. After focal ischemia, a rat tends to swing to the contralateral (left) side.

Perfusion and Collection of Brains. At twenty-eight days (Day 28) after MCAO, rats were anesthetized deeply with ketamine/xylazine (91 mg/kg ketamine, 9 mg/kg xylazine, respectably). After the rats were in the deep anesthetized stage, the rats were perfused transcardially with normal saline (with heparin 2 unit/mL) followed by 4% paraformaldehyde. Brains were removed and stored in 4% paraformaldehyde for overnight then changed to 1xPBS and stored at 0-4° C. The brain samples will be used for histology and immunofluorescence staining to evaluate brain infarct size, neurogenesis, neovascularization, and various other markers of GDF11 activity.

### Results

Clinical Observations and Survival. No death was observed for this study. All the animals appeared normal.

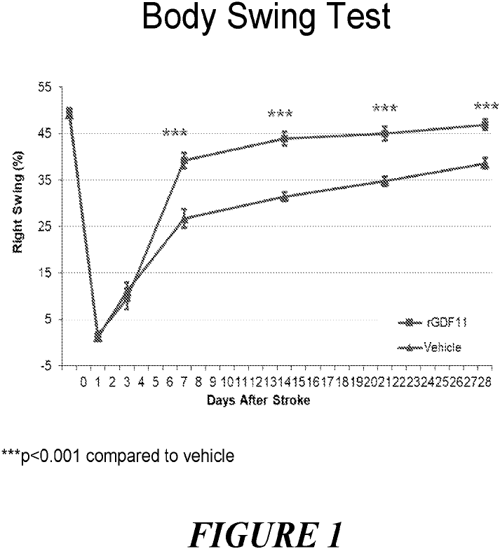

Behavioral Test 1 (Forelimb Placing Test). The results of the forelimb placing test are depicted in FIG. 1. There were no differences between the 2 groups before treatment started. As can be seen in FIG. 1, animals that received rGDF11 i.p. at 1 mg/kg, on Day 3 through Day 16, showed superior recovery compared to vehicle-treated animals on Day 14 (p<0.05); a trend for Day 21 (p=0.060). No statistically significant differences were observed on Day 7 or Day 28.

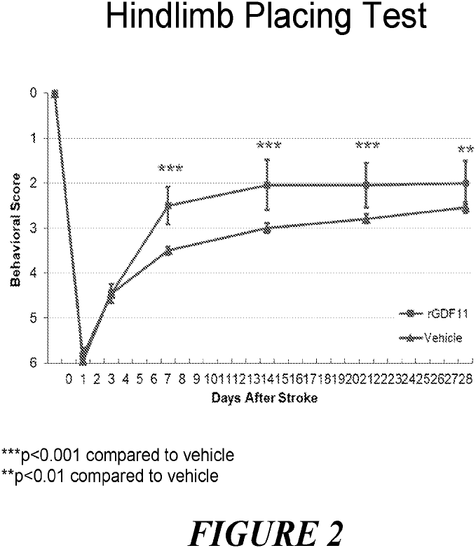

Behavioral Test 2 (Hindlimb Placing Test). The results of the hindlimb placing test are depicted in FIG. 2. There were no differences between the 2 groups before treatment started. As can be seen in FIG. 2, animals that received rGDF11 i.p. at 1 mg/kg, at Day 3 through Day 16, showed superior recovery compared to vehicle-treated animals on Day 7 (p<0.001), Day 14 (p<0.001), Day 21 (p<0.001) and Day 28 (p<0.01). No statistically significant differences were observed on Day 3.

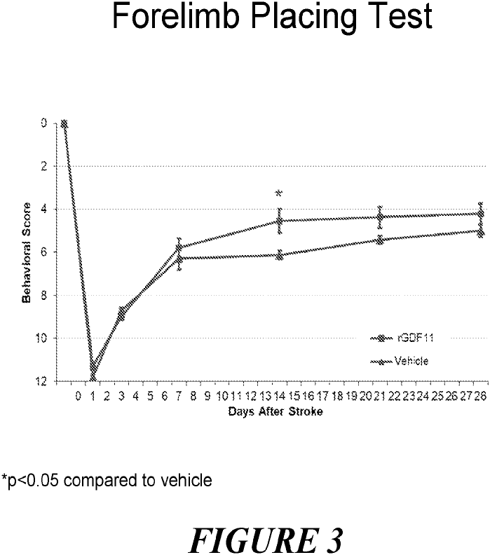

Behavioral Test 3 (Body Swing Test). The results of the body swing test are depicted in FIG. 3. There were no differences between the 2 groups before treatment started. As can be seen in FIG. 3, animals that received rGDF11 i.p. at 1 mg/kg, at Day 3 through Day 16, showed superior recovery compared to vehicle-treated animals Day 7 (p<0.001), Day 14 (p<0.001), Day 21 (p<0.001) and Day 28 (p<0.001). No statistically significant differences were observed on Day 3 or Day 5.

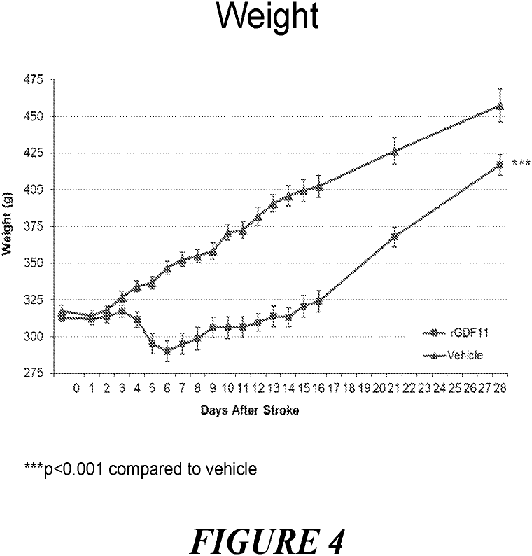

Weight Change Test. The results of the body weight test are depicted in FIG. 4. There were no differences between the 2 groups before treatment started. As can be seen in FIG. 4, animals that received rGDF11 i.p. at 1 mg/kg, at Day 3 through Day 16 showed significant reduction of weight compared to the vehicle-treated animals (p<0.001). Body weight in the rGDF11 treated animals was reduced by 7.03% on the third day post-surgery relative to their weights immediately after surgery. Thereafter, rGDF1 1 treated animals gained weight at a rate comparable to the vehicle treated animals.

Conclusions. Middle cerebral artery occlusion (MCAO) was made in mature male Sprague-Dawley rats, resulting in focal unilateral cerebral infarct. Vehicle (1 ml/kg) or rGDF11 (1 mg/kg, 1 ml/kg) was given i.p. from 3 days to 16 days after MCAO. Behavioral assessments of sensorimotor function: limb placing tests were made at prior to MCAO, 1 day, 3 days, 7 days, 14 days, 21 days and 28 days after MCAO. The body swing test was performed on the same schedule as the limb placing tests. As can be seen by the results of this experiment, the study demonstrated significant enhancement of sensorimotor performance after stroke, especially in body swing, hindlimb placing, and forelimb placing tests for the rGDF11treated group. The magnitude and durability of these improvements were greatest in the body swing and hindlimb placing tests. In addition, an initial reduction in body weight was observed for the rGDF11 treated animals, resulting in a 7.03% decrease on the third day post-surgery relative to their weights immediately after surgery. However, the rGDF11 treated animals gained weight at a rate comparable to the vehicle treated animals thereafter although the decreased body weight for the rGDF11 treated animals relative to the vehicle treated controls was maintained throughout the duration of the study.

### Example 2

The purpose of this example was to evaluate the therapeutic effect of a GDF11 molecule administration regimen consisting of a single dose of rGDF11 administered once daily (QD) for 7 days starting at one day post-occlusion in a rat permanent Middle Cerebral Artery Occlusion Model (pMCAO) study. Body motor function was measured out to 28 days post-occlusion.

The pMCAO methodology model, animals, animal preparation were the same as described in Example 1.

Dosing. Animals were dosed starting on day 1 post the surgical occlusion of the MCA with dosing continuing daily, through day 7 after the MCAO. Animals received vehicle at 1 ml/kg or rGDF11 i.p. 1 mg/kg (1 ml/kg) once daily.

Body weight, blood collection, functional behavioral tests, limb placement, body swing test, and perfusion and collection of brains were measured or performed as described in Example 1.

### Results:

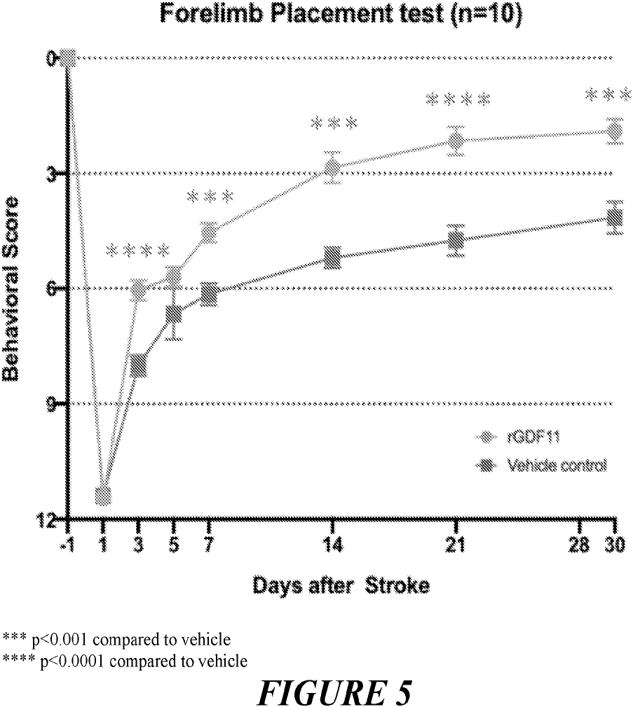

Behavioral Test 1 (Forelimb Placing Test). The result of the forelimb placing test are depicted in FIG. 5. There were no differences between the 2 groups before treatment started. Animals that received rGDF11 i.p. at 1 mg/kg beginning Day 1 through Day 7 showed superior recovery compared to vehicle-treated animals on Day 3 (p<0.0001), Day 7 (p<0.001), Day 14 (p<0.001), Day 21 (p<0.0001), and Day 30 (p<0.001). No statistically significant differences were observed on Day 5.

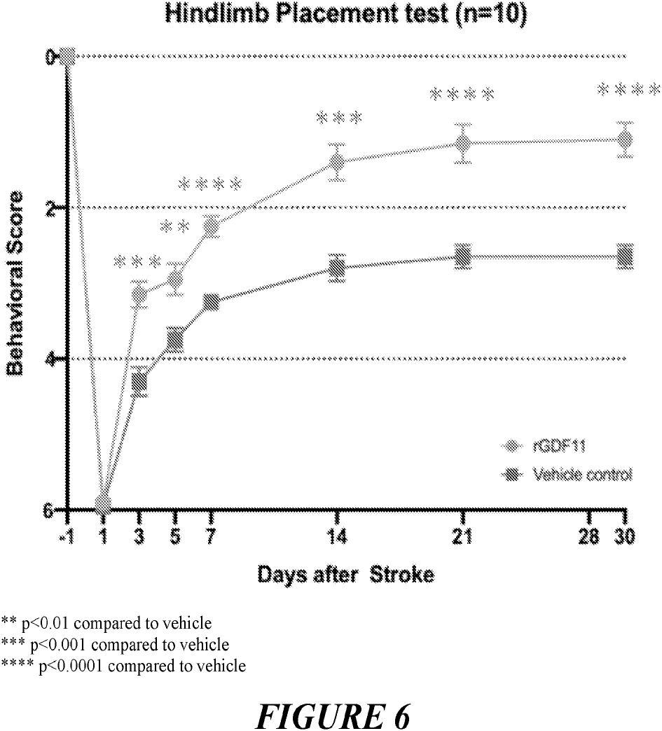

Behavior Test 2 (Hindlimb Placing Test). The results of the hindlimb placing test are depicted in FIG. 6. There were no differences between the 2 groups before treatment started. Animals that received rGDF11 i.p. at 1 mg/kg beginning Day 1 through Day 7 showed superior recovery compared to vehicle-treated animals on Day 3 (p<0.001), Day 5 (p<0.01), Day 7 (p<0.0001), Day 14 (p<0.001), Day 21 (p<0.0001), and Day 30 (p<0.0001).

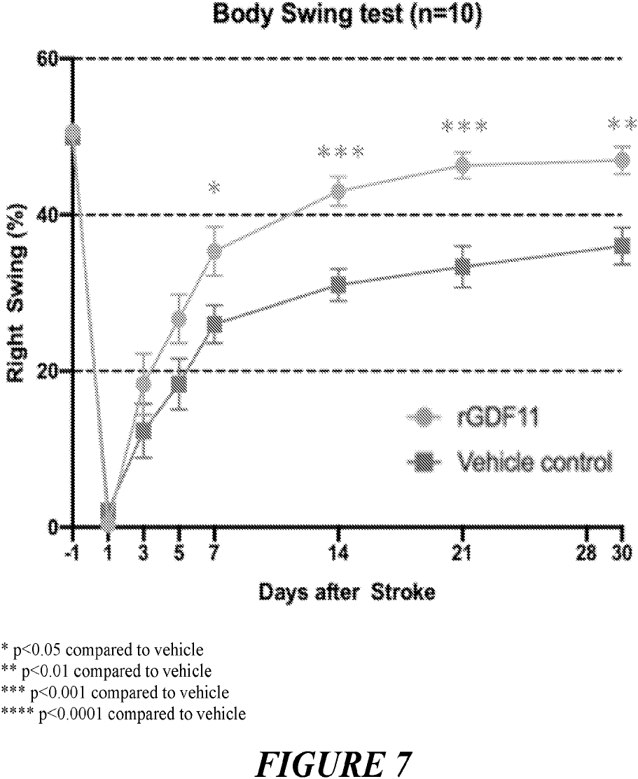

Behavioral Test 3 (Body Swing Test). The results of the body swing test are depicted in FIG. 7. There were no differences between the 2 groups before treatment started. Animals that received rGDF11 i.p. at 1 mg/kg beginning Day 3 through Day 7 showed superior recovery compared to vehicle-treated animals Day 7 (p<0.05), Day 14 (p<0.001), Day 21 (p<0.001), and Day 30 (p<0.01). No significant differences were observed on Day 3 or Day 5.

### Conclusions.

Middle cerebral artery occlusion (MCAO) was made in mature male Sprague-Dawley rats, resulting in focal unilateral cerebral infarct. Vehicle (1 ml/kg) or rGDF1 1 (1 mg/kg, 1 ml/kg) was given i.p. from 1 days to 7 days after MCAO. Behavioral assessments of sensorimotor function: limb placing tests were made at prior to MCAO, 1 day, 3 days, 7 days, 14 days, 21 days and 30 days after MCAO. The body swing test was performed on the same schedule as the limb placing tests. As can be seen by the results of this experiment, the study demonstrated showed durable sensorimotor function improvement after stroke, especially in hindlimb placing, forelimb placing, and body swing tests for the rGDF11 treated group. Significant improvement in limb placement tests was observed after two doses. Sensorimotor function improvement maintained for at least 30 days post occlusion.

### Example 3

The purpose of this example was to evaluate the therapeutic effect of a GDF11 molecule administration regimen consisting of single daily doses of rGDF11 administered for durations varying from 1 to 7 days (i.e., treating for 1, 3, 5, or 7 days) starting at one (1) day post-occlusion in a rat permanent Middle Cerebral Artery Occlusion Model (pMCAO) study. Body motor function was measured out to 14 days post-occlusion.

The pMCAO methodology model, animals, animal preparation were the same as described in Example 1.

Dosing. Animals were dosed starting on day 1 post surgical occlusion of the MCA with dosing continuing daily for 1, 3, 5, or 7 days after the MCAO. Animals received vehicle at 1 ml/kg or rGDF11 i.p. 1 mg/kg (1 ml/kg) once daily.

Body weight, blood collection, functional behavioral tests, limb placement, body swing test, and perfusion and collection of brains were measured or performed as described in Example 1.

### Results:

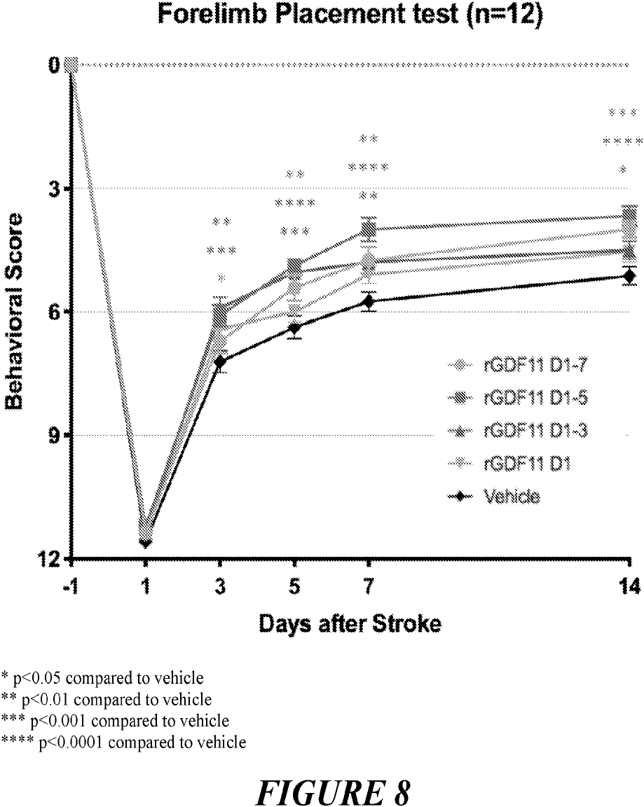

Behavioral Test 1 (Forelimb Placing Test). The result of the forelimb placing test are depicted in FIG. 8 for animals treated with rGDF1 1 for 1, 3, 5, and 7 days, and animals treated with the vehicle. There were no differences between the 5 groups before treatment started. Animals that received rGDF11 i.p. at 1 mg/kg on only Day 1 showed superior recovery time compared to vehicle-treated animals on Day 3 (p<0.05), but with no statistically significant differences observed on Day 5, Day 7, or Day 14. Animals that received rGDF11 i.p. at 1 mg/kg through Day 3 showed superior recovery time compared to vehicle-treated animals on Day 3 (p<0.001), Day 5 (p<0.001), Day 7 (p<0.01), and Day 14 (p<0.05). Animals that received rGDF11 i.p. at 1 mg/kg through Day 5 showed superior recovery time compared to vehicle-treated animals on Day 3 (p<0.01), Day 5 (p<0.0001), Day 7 (p<0.0001), and Day 14 (p<0.0001). Animals that received rGDF11 i.p. at 1 mg/kg through Day 7 showed superior recovery time compared to vehicle-treated animals on Day 5 (p<0.01), Day 7 (p<0.01), and Day 14 (p<0.001), with no statistically significant differences observed on Day 3.

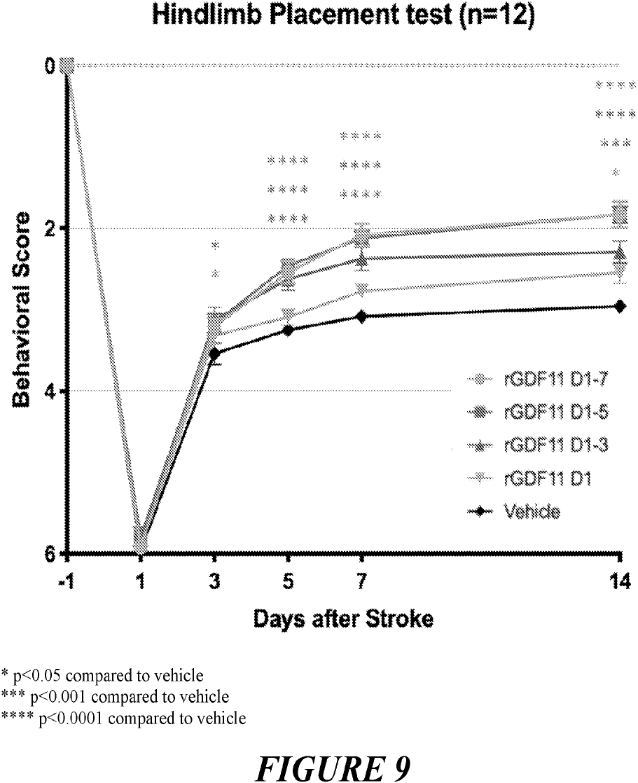

Behavior Test 2 (Hindlimb Placing Test). The results of the hindlimb placing test are depicted in FIG. 9. There were no differences between the 5 groups before treatment started. Animals that received rGDF11 i.p. at 1 mg/kg on only Day 1 showed superior recovery time compared to vehicle-treated animals on Day 3 (p<0.05) and Day 14 (p<0.05), with no statistically significant differences observed on Day 5 or Day 7. Animals that received rGDF11 i.p. at 1 mg/kg through Day 3 showed superior recovery time compared to vehicle-treated animals on Day 3 (p<0.05), Day 5 (p<0.0001), Day 7 (p<0.0001), and Day 14 (p<0.001). Animals that received rGDF11 i.p. at 1 mg/kg through Day 5 showed superior recovery time compared to vehicle-treated animals on Day 5 (p<0.0001), Day 7 (p<0.0001), and Day 14 (p<0.0001), with no statistically significant difference observed on Day 3. Animals that received rGDF11 i.p. at 1 mg/kg through Day 7 showed superior recovery time compared to vehicle-treated animals on Day 5 (p<0.0001), Day 7 (p<0.0001), and Day 14 (p<0.0001), with no statistically significant difference observed on Day 3.

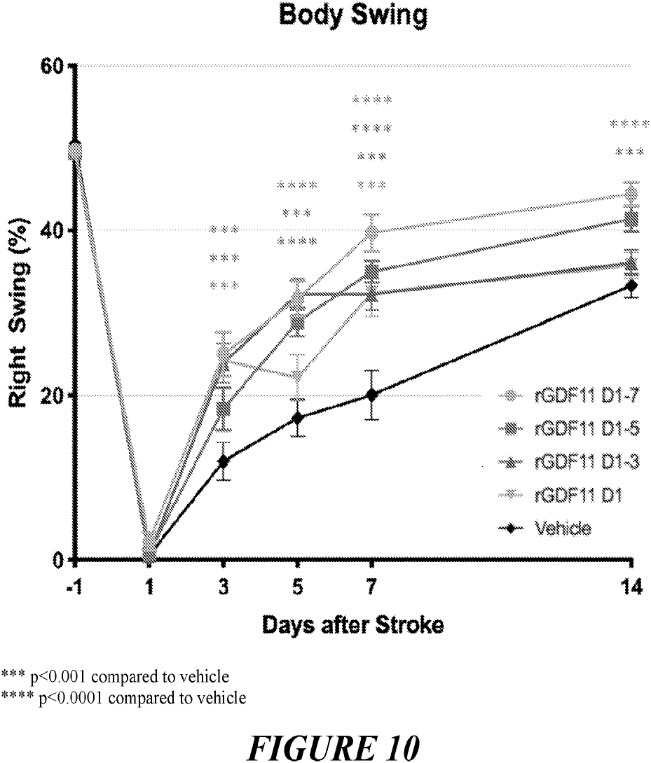

Behavioral Test 3 (Body Swing Test). The results of the body swing test are depicted in FIG. 10. There were no differences between the 5 groups before treatment started. Animals that received rGDF11 i.p. at 1 mg/kg on only Day 1 showed superior recovery time compared to vehicle-treated animals on Day 3 (p<0.001) and Day 7 (p<0.001), with no statistically significant differences observed on Day 5 or Day 14. Animals that received rGDF11 i.p. at 1 mg/kg through Day 3 showed superior recovery time compared to vehicle-treated animals on Day 3 (p<0.001), Day 5 (p<0.0001), and Day 7 (p<0.001), with no statistically significant difference observed on Day 14. Animals that received rGDF11 i.p. at 1 mg/kg through Day 5 showed superior recovery time compared to vehicle-treated animals on Day 5 (p<0.001), Day 7 (p<0.0001), and Day 14 (p<0.001), with no statistically significant difference observed on Day 3. Animals that received rGDF11 i.p. at 1 mg/kg through Day 7 showed superior recovery time compared to vehicle-treated animals on Day 3 (p<0.001), Day 5 (p<0.0001), Day 7 (p<0.0001), and Day 14 (p<0.0001).

### Conclusion.

Shorter durations of rGDF 11 treatment yielded transient but significant sensorimotor function improvement was observed for only 1 or 3 days. Specifically, for the group that received only single dose rGDF11 treatment at 1 day after occlusion, significant improvement was observed at Day 3 for all three assessments, significant improvement in Body Swing outcome at Day 7, and significant improvement in Hindlimb Placement Test at Day 14. A 5-day treatment regimen was not inferior to a 7-day treatment regimen. The 5 and 7-day treatment periods showed surprisingly durable therapeutic effects.

### Example 4

The purpose of this example was to evaluate the therapeutic effect of a GDF11 molecule administration regimen for a range of doses (0.1, 0.5, 1.0, 2.0, and 4.0 mg/kg) in a dosing regimen where rGDF11 is administered for starting at one (1) day post-occlusion in a rat permanent Middle Cerebral Artery Occlusion Model (pMCAO) study. Body motor function was measured out to 28 days post-occlusion.

The pMCAO methodology model, animals, animal preparation were the same as described in Example 1.

Dosing. Animals were dosed starting on day 1 post surgical occlusion of the MCA with dosing continuing daily for 5 days at 5 different doses: 0.1, 0.5, 1, 2, and 4 mg/kg after the MCAO. Animals received vehicle at 1 ml/kg or rGDF11 i.p. 1 mg/kg (1 ml/kg) once daily.

Body weight, blood collection, functional behavioral tests, limb placement, body swing test, and perfusion and collection of brains were measured or performed as described in Example 1.

### Results:

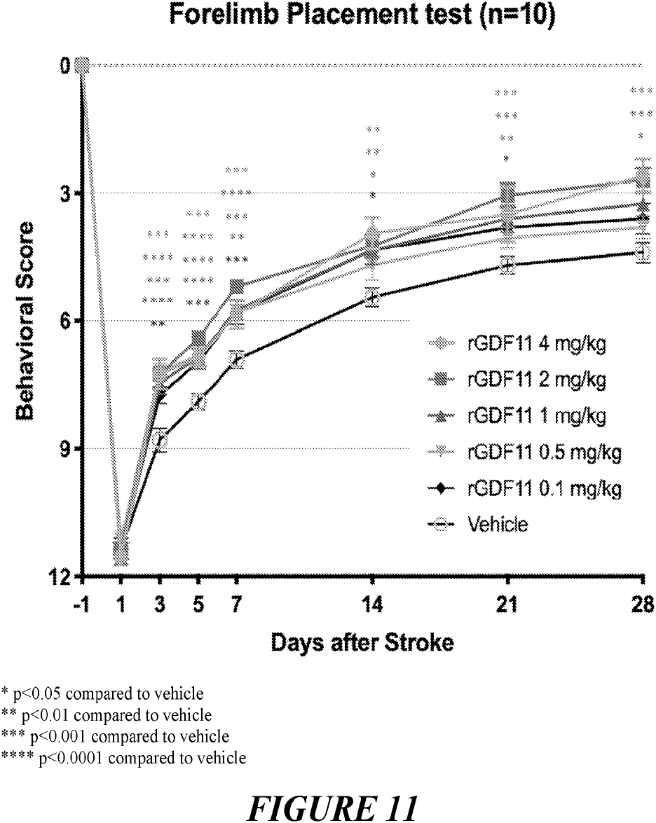

Behavioral Test 1 (Forelimb Placing Test). The result of the forelimb placing test are depicted in FIG. 11 for animals treated with at doses of 0.1, 0.5, 1, 2, and 4 mg/kg, and animals treated with the vehicle. There were no differences between the 6 groups before treatment started. Animals that received rGDF11 i.p. at 0.1 mg/kg through Day 5 showed superior recovery time compared to vehicle-treated animals on Day 3 (p<0.01), Day 5 (p<0.001), Day 7 (p<0.001), Day 14 (p<0.05), and Day 21 (p<0.05), with no statistically significant differences observed on Day 28. Animals that received rGDF11 i.p. at 0.5 mg/kg through Day 5 showed superior recovery time compared to vehicle-treated animals on Day 3 (p<0.0001), Day 5 (p<0.0001), Day 7 (p<0.01), and Day 14 (p<0.05), with no statistically significant differences observed on Day 21 or Day 28. Animals that received rGDF11 i.p. at 1.0 mg/kg through Day 5 showed superior recovery time compared to vehicle-treated animals on Day 3 (p<0.001), Day 5 (p<0.0001), Day 7 (p<0.001), Day 14 (p<0.05), Day 21 ((p<0.01), and Day 28 (p<0.05). Animals that received rGDF11 i.p. at 2.0 mg/kg through Day 5 showed superior recovery time compared to vehicle-treated animals on Day 3 (p<0.0001), Day 5 (p<0.0001), Day 7 (p<0.001), Day 14 (p<0.01), Day 21 ((p<0.001), and Day 28 (p<0.001). Animals that received rGDF11 i.p. at 4.0 mg/kg through Day 5 showed superior recovery time compared to vehicle-treated animals on Day 3 (p<0.001), Day 5 (p<0.001), Day 7 (p<0.001), Day 14 (p<0.01), Day 21 ((p<0.001), and Day 28 (p<0.001).

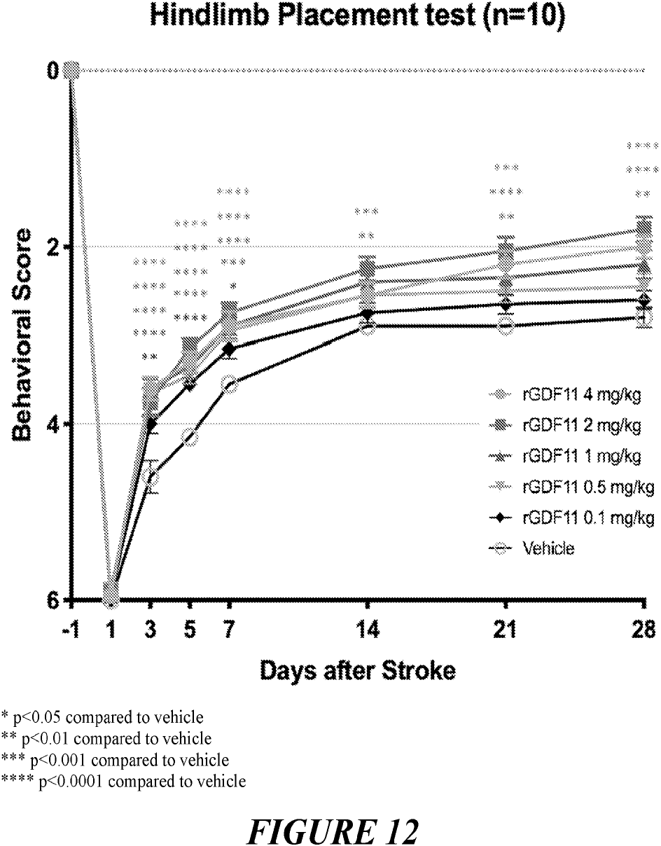

Behavior Test 2 (Hindlimb Placing Test). The results of the Hindlimb Placing Test are depicted in FIG. 12 for animals treated with at doses of 0.1, 0.5, 1, 2, and 4 mg/kg, and animals treated with the vehicle. There were no differences between the 6 groups before treatment started. Animals that received rGDF11 i.p. at 0.1 mg/kg through Day 5 showed superior recovery time compared to vehicle-treated animals on Day 3 (p<0.01), Day 5 (p<0.0001), and Day 7 (p<0.05), with no statistically significant differences observed on Day 14, Day 21, or Day 28. Animals that received rGDF11 i.p. at 0.5 mg/kg through Day 5 showed superior recovery time compared to vehicle-treated animals on Day 3 (p<0.0001), Day 5 (p<0.0001), and Day 7 (p<0.001), with no statistically significant differences observed on Day 14, Day 21, or Day 28. Animals that received rGDF11 i.p. at 1.0 mg/kg through Day 5 showed superior recovery time compared to vehicle-treated animals on Day 3 (p<0.0001), Day 5 (p<0.0001), Day 7 (p<0.0001), Day 14 (p<0.01), Day 21 ((p<0.01), and Day 28 (p<0.01). Animals that received rGDF11 i.p. at 2.0 mg/kg through Day 5 showed superior recovery time compared to vehicle-treated animals on Day 3 (p<0.0001), Day 5 (p<0.0001), Day 7 (p<0.0001), Day 14 (p<0.001), Day 21 ((p<0.0001), and Day 28 (p<0.0001). Animals that received rGDF11 i.p. at 4.0 mg/kg through Day 5 showed superior recovery time compared to vehicle-treated animals on Day 3 (p<0.0001), Day 5 (p<0.0001), Day 7 (p<0.0001), Day 21 ((p<0.001), and Day 28 (p<0.0001), with no statistically significant differences observed on Day 14.

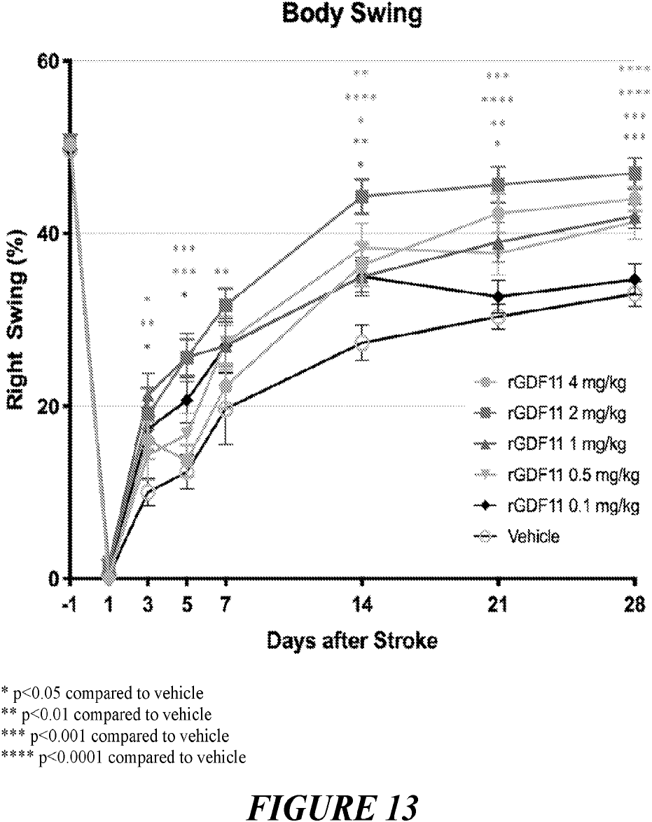

Behavioral Test 3 (Body Swing Test). The results of the body swing test are depicted in FIG. 13. There were no differences between the 6 groups before treatment started. Animals that received rGDF11 i.p. at 0.1 mg/kg through Day 5 showed improved recovery time compared to vehicle-treated animals on Day 3 (p<0.05), Day 5 (p<0.05), and Day 14 (p<0.05), with no statistically significant differences observed on Day 7, Day 21, and Day 28. Animals that received rGDF11 i.p. at 0.5 mg/kg through Day 5 showed improved recovery time compared to vehicle-treated animals on Day 14 (p<0.01), Day 21 (p<0.05), and Day 28 (p<0.001), with no statistically significant differences observed on Day 3, Day 5, or Day 7. Animals that received rGDF11 i.p. at 1.0 mg/kg through Day 5 showed superior recovery time compared to vehicle-treated animals on Day 3 (p<0.01), Day 5 (p<0.001), Day 14 (p<0.05), Day 21 ((p<0.01), and Day 28 (p<0.001), with no statistically significant differences observed on Day 7. Animals that received rGDF11 i.p. at 2.0 mg/kg through Day 5 showed superior recovery time compared to vehicle-treated animals on Day 3 (p<0.05), Day 5 (p<0.001), Day 7 (p<0.01), Day 14 (p<0.0001), Day 21 ((p<0.0001), and Day 28 (p<0.0001). Animals that received rGDF11 i.p. at 4.0 mg/kg through Day 5 showed superior recovery time compared to vehicle-treated animals on Day 14 (p<0.01), Day 21 ((p<0.001), and Day 28 (p<0.0001), with no statistically significant differences observed on Day 3, Day 5, or Day 7.

### Conclusion.

rGDF11 treatment improved sensorimotor function recovery over a wide dose-range. Efficacy in all treatment groups at various time points was observed with a dose range of 0.1 - 4.0 mg/kg rGDF11 with a 5-day daily treatment. In particular, in animals administered doses of 0.1 mg/kg and 0.5 mg/kg, forelimb and hindlimb improvement after 7 days was either not observed or was substantially lower in magnitude. No statistically significant improvement in either the forelimb placing test or hindlimb placing test at the final day of the experiment (Day 28). However, those animals administered 1.0 mg/kg, 2.0 mg/kg, and 4.0 mg/kg rGDF11 surprisingly showed statistically significant improvement in forelimb placing test and hindlimb placing test through Day 28. Animals with doses of 1.0 mg/kg, 2.0 mg/kg, and 4.0 mg/kg rGDF11 showed a surprising and remarkable long-term therapeutic effect in comparison to the lower 0.1 mg/kg and 0.5 mg/kg doses.

### Example 5

The purpose of this example was to evaluate the therapeutic effect of a GDF11 molecule administration regimen consisting of a single dose of rGDF11 administered 30 minutes post-ICH, and then once daily for seven (7) days (q24 x 7) in a C57B16/j mouse model.

### ICH Mouse Model.

Eleven-week-old male C57BL/6J mice (Jackson Laboratory, Bar Harbor, ME) were housed on a 12-hour light/dark cycle in standard acrylic cages with ad libitum access to food and water. In each experiment, mice were randomized to treatment or vehicle groups before injury.

Animals were treated with blinded concealment. All procedures and assessments were performed in blinded fashion.

Intrastriatal collagenase injection was used to induce ICH in mice. The trachea was intubated after anesthesia induction with 4.6% isoflurane, and the lungs were mechanically ventilated with 1.5% isoflurane in a mixture of 30%/70% O2/N2. Rectal temperature was maintained at 37° C. ± 0.2° C. by circulating warm water in an underbody waterbed. The head of the animal was secured in a stereotactic frame. A midline scalp incision was made. After exposing the skull, a burr hole was created 2.2 mm left lateral to bregma, and a 0.5 µL syringe needle (Hamilton, Reno, NV, USA) was advanced to a depth of 3 mm from cortex. Type IV-S Clostridial collagenase (Sigma, St. Louis, MO, USA) was injected over 2 minutes (0.075 U in 0.4 µL normal saline). After closing the incision, animals were allowed to recover to spontaneous ventilation before being extubated and given free access to food and water.

Animals receive vehicle or rGDF11, i.p., starting at 30 minutes after ICH and continued daily for 7 days. All test articles were labeled by codes. Both the surgeon and the researcher who carried out the behavioral assessments were blinded to the treatment assignments.

100 µl of A (1.0 mg/kg rGDF11) or B (Vehicle) solution interperitoneal (IP) injections were administered daily at 30 minutes after ICH and q24 x 7 days thereafter.

Neurobehavioral assessment. Animals were randomized to vehicle (n = 22) or (n = 22) rGDF11 groups, and evaluated for mortality, NeuroSeverity Score (NSS), Rotarod Latency (RR), Average Speed 7 days post-treatment (CatWalk), and Forelimb Base of Support 7 days post-treatment (CatWalk).

NeuroSeverity Score. Animals were evaluated based on their behavior in seven categories including spontaneous activity, symmetry, climbing, balance and coordination, body proprioception, vibrissae touch, and tactile response. The detailed scoring criteria is in Appendix A. for the NSS, 21 = normal, 3 = dead. These tests were performed before ICH surgery (Day 0), and at 1, 2, 3, 4, 5, 6, 7, 14, 21, and 28 day(s) after ICH. (Day 1 = day of ICH).

Rotarod testing. An automated rotarod (Ugo Basile, Comerio, Italy) was used to assess the effects of therapeutic intervention on vestibulomotor function. On the day before injury, mice underwent 2 consecutive con- ditioning trials at a set rotational speed of 16 revolutions/minute for 60 seconds, followed by 3 additional trials at accelerating (4-40) rotational speeds. The average time lapse to fall from the rotating cylinder in the second set of trials was recorded as baseline latency. To assess motor outcome, mice underwent rotarod testing on Days 1-7, 14, 21, and 28 after injury. On each day, mice underwent 3 trials with an inter-trial interval of 15 minutes. Average latency to fall from the rod was recorded.

Throughout all rotarod testing, under the following circumstances a trial is manually terminated and latency until the termination is recorded for the particular trial: 1) the mouse is unable to keep running on the rotating rod and hold the rod still for two consecutive revolutions; 2) 20 seconds after the rotating speed reaches the max speed of 40 rpm.

CatWalk Analysis. Fine characteristics of voluntary, overground locomotion were collected and analyzed using the CatWalk XT system (Noldus Information Technology; Leesburg, VA). Illuminated footprint detection of all four limbs occurred via video recording of mice as they traversed an internally lit glass plate. In this setting, paw contact with the floor appears bright while the body of the mouse appears dark. An experimenter, blind to treatment groups, performed all procedures. Mice were acclimated to the room and system, then trained to cross the walkway continuously prior to baseline data collection. A successful run required continuous locomotion across the walkway without stopping or sniffing. Automated labeling of footprints occurred using the CatWalk XT software (v10.6). Visual inspection and manual correction of incorrectly labeled footprints occurred. Automated gait analysis occurred using CatWalk XT software (v10.6) and the following parameters of interest were averaged across three successful runs and analyzed: average speed, base of support (forelimbs, hindlimbs), cadence, regularity index, single-limb support, variation. Outlier scores exceeding 2 standard deviations from the mean excluded from analyses (n=1 per group). Between-group comparisons occurred at 7d post-injury using independent samples t-tests.

With regard to brain/tissue collection for the ICH study, the animals were sacrificed on day 28 post-injury.

Statistical analyses: Multiple Kolmogorov-Smimov tests or two-way analysis of variance (ANOVA) with repeated measures was used to compare neuroseverity scores, rotarod latencies, and Catwalk analysis, with time as the repeated variable. Bonferroni correction was used for the repeated measures technique in ANOVA. A p value < 0.05 was considered statistically significant. All values are expressed as means ± standard error. Statistical analysis was performed using SPSS.

### Mortality.

ICH injection resulted in 3 deaths 24 hours after injury (#9, 11, 14). The vehicle group resulted in 2 deaths at 24 h (#10, 35), 4 death on Day6 (#26, 28, 29, 36), 1 death on Day 10 (#34).

| Group | Total | Death (Before Treatment) | Death (After Treatment) | No deficit (NSS) * | Final** (in analysis) |
|---|---|---|---|---|---|
| rGDF11 | 22 | 0 | 3/22 | 0 | 22 |
| Vehicle | 22 | 0 | 7/22 | 0 | 22 |

* All animals with ICH had behavioral deficit after injury
** Dead animals received minimal score of 3 in NSS and 0 in RR

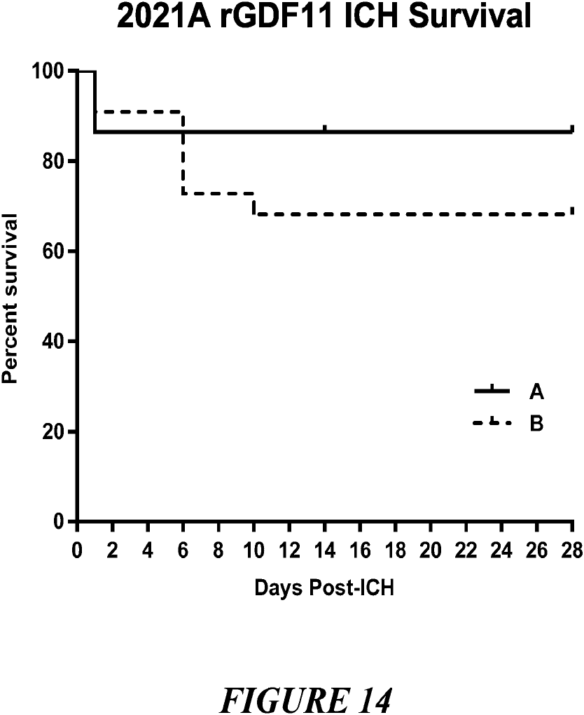

FIG. 14 depicts the percent survival of Group A (rGDF11 treatment group) and Group B (vehicle group) for C57B16/j mice as a function of days post-ICH at Day 0. Survival analysis was performed using log rank (Prism 7.0), p = 0.1893. Group A had three deaths within 24 hours of injury, and no deaths for the rest of the following 28 days. Group B had two deaths within 24 hours of injury, and subsequently four deaths on Day 6 and one on Day 10. Group B trended toward decreased survival.

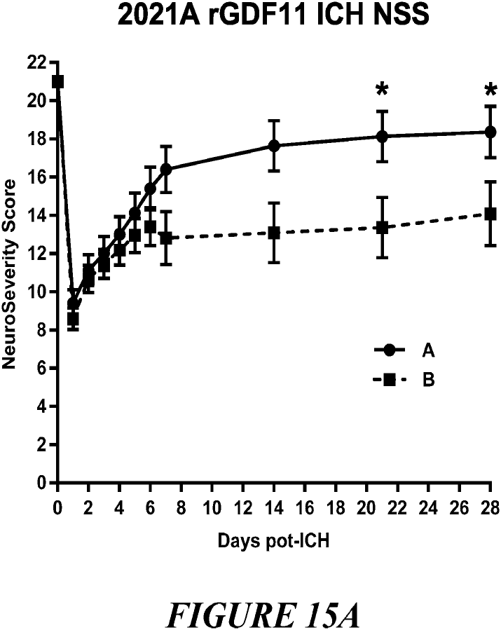

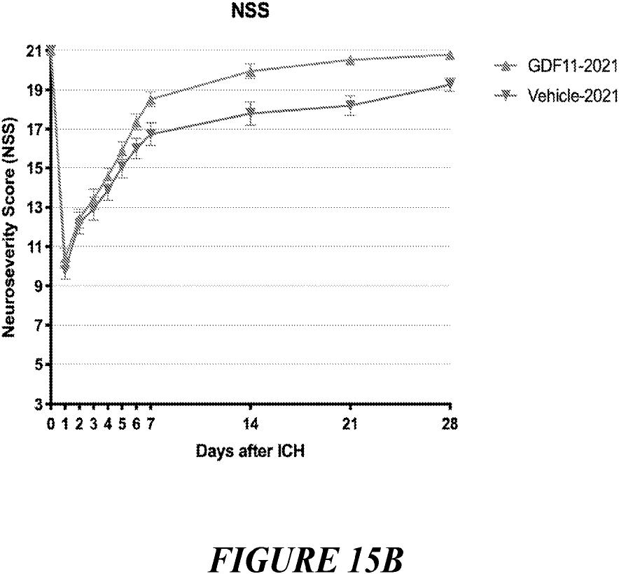

FIG. 15A depicts the post-ICH NeuroSeverity Score over the days Group A and Group B following injection, including dead animals. The NeuroSeverity Score was assessed daily on day 0, 1, 2, 3, 4, 5, 6, 7, 14, 21, 28. Two Way analysis of variance (ANOVA) with repeated measures performed using SPSS, p=0.121. The NeuroSeverity Score of Group A showed measurable improvement as compared to Group B at Day 7, Day 14, Day 21, and Day 28. The daily independent t-Test showed statistically independent results at Day 21 (p=0.048) and Day 28 (0.019).

FIG. 15B depicts the NeuroSeverity Score over the days post-ICH of Group A and Group B following injection, excluding dead animals where n = 19 for Group A and n = 15 for Group B. Two Way analysis of variance (ANOVA) with repeated measurements with Bonferroni’s correction performed using Graphpad Prism software. The NeuroSeverity Score of Group A showed measurable improvement as compared to Group B at Day 5, Day 7, Day 14, Day 21, and Day 28. The Two Way ANOVA showed statistically independent results at Day 21 (p=0.006) and Day 28 (p=0.008).

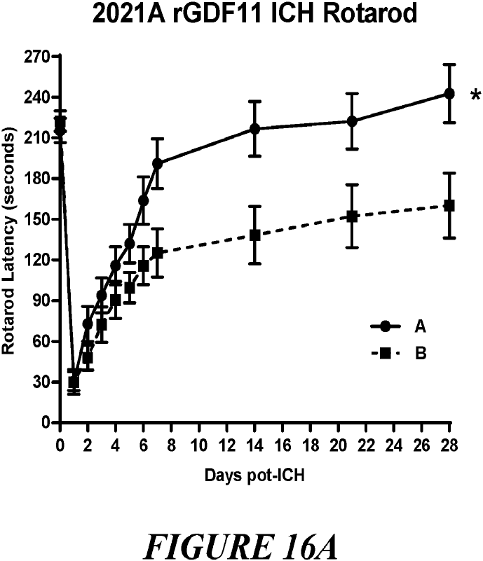

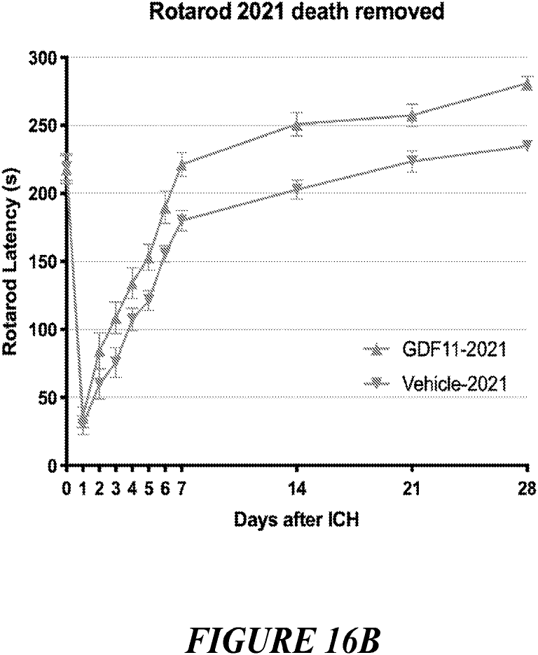

FIG. 16A depicts a plot of rotarod latency as a function of days post-ICH injury for Group A and the Group B, including dead animals. Survival Analysis was performed using log rank (Prism 7.0), p = 0.1893. Daily rotarod tests were performed on Days 0, 1, 2, 3, 4, 5, 6, 7, 14, 21, and 28. Two Way ANOVA with repeated measurements performed using SPSS, p = 0.024. Beginning at Day 4, Group A had improved rotarod latency, which continued through Day 28. At Days 14 and 28 Group B showed a statistically significant (p<0.05) reduction in rotarod latency as compared to Group A (two tailed ANOVA 0.024). At Day 6, Day 7, Day 14, Day 21, and Day 28, the rotarod latency for Group A was within statistical error of the pre-injury Rotarod Latency. Group B did not improve to the pre-injury rotarod latency level over 14 days. At Day 21, and Day 28, the rotarod latency for Group B was within statistical error of the pre-injury Rotarod Latency.

FIG. 16B depicts a plot of rotarod latency as a function of days post-ICH injury for Group A and the Group B, excluding dead animals. Daily rotarod tests were performed on Days 0, 1, 2, 3, 4, 5, 6, 7, 14, 21, and 28. Statistical analysis was performed using multiple Kolmogorov-Smimov tests or Two-way ANOVA with correction. Beginning at Day 4, Group A had improved rotarod latency, which continued through Day 28. At Days 7, 14, and 28, Group B (Vehicle) showed a statistically significant (p<0.05) reduction in rotarod latency as compared to Group A (GDF11). At Day 6, Day 7, Day 14, Day 21, and Day 28, the rotarod latency for Group A was within statistical error or greater than that of the pre-injury Rotarod Latency. Group B improved to within statistical error of the pre-injury rotarod latency level after Day 14.

A CatWalk assessment was performed the mice in Group A and Group B on Day 0, Day 2, and Day 7. The CatWalk test measured average speed and forelimb base of support for all groups.

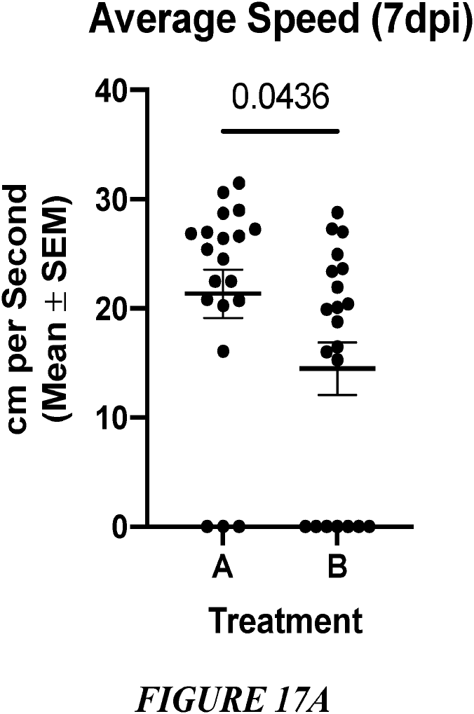

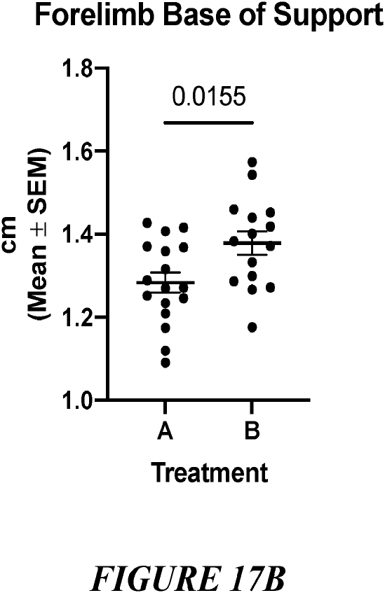

FIGS. 17A and 17B depicts the locomotor performance of Group A compared to Group B. FIG. 17A shows the average speed in in centimeters per second seven (7) days after first treatment. FIG. 17B depicts the forelimb base of support for the rGDF11 administered group (Group A) compared to the vehicle group (Group B).

A systematic difference between groups was noted as a function of therapeutic assignment. Group B trended toward non-survival. At days 21 and 28, there was a significant (p<0.05) reduction in NSS in Group B (two tailed ANOVA 0.121). At days 7, 14, 21, 28 there was a significant (p<0.05) reduction in Rotarod in Group B (two tailed ANOVA 0.024). Using Catwalk analysis, there was a significant reduction in average gait speed in Group B (p=0.043). Group B showed a statistically significant reduction in average gait speed in Group B (p=0.043). Group B also showed a statistically significant increase in forelimb base of support (p=0.015).

### Example 6

The purpose of this example was to evaluate the neurogenesis of five days GDF 11 treatment at different doses on stroke recovery for 24 days post treatment. Neurogenesis was measured using the Sox2 multipotential neural stem cell marker.

The pMCAO methodology model, animals, animal preparation were the same as described in Example 1.

Dosing. Animals were dosed starting on day 1 post surgical occlusion of the MCA with dosing continuing daily for 5 days at 5 different doses: 1, 2, and 4 mg/kg after the MCAO. Animals received vehicle at 1 ml/kg or rGDF11 i.p. 1 mg/kg (1 ml/kg) once daily.

Imaging: Rats were transcardially perfused with PBS followed by 4% PFA. Brains were extracted, washed with PBS and then cryopreserved with 20% sucrose. After cryopreserving, the brains were embedded in optimal cutting temperature (OCT) compound and stored at -20° C.

Free floating 50 µm sections were collected using a Leica cryostat. Sections from 6 rats were spatially matched and prepared for immunofluorescence staining. Antigen retrieval was performed using 1x citrate buffer (pH 6.0) at 90° C. for 10 minutes. Brain sections were then washed with PBS and blocked for 1 hour in blocking buffer (1x PBS, 0.5% Triton X-100, and 10% Normal Donkey Serum) at room temperature. Samples were washed with wash buffer (1x PBS 0.5% Triton X-100) prior to incubation with primary antibody. Primary antibody (Rabbit polyclonal to SOX2, Abcam; ab97959) was diluted 1:200 in antibody dilution buffer (1x PBS, 1% BSA, and 0.5% Triton X-100) and incubated overnight at 4° C. with gentle agitation. Following primary antibody incubation, sections were washed three times with wash buffer and incubated for 1 hour at room temperature with secondary antibody (Donkey anti-Rabbit IgG Alexa Fluor 488, Invitrogen; A-21206) diluted 1:2000 in antibody dilution buffer. Secondary antibody was then removed, and sections were incubated with 300 nM of DAPI, a nuclear staining reagent (Invitrogen; D3571) in PBS for 10 minutes before washing 3 times with wash buffer. Slides Sections were then placed on microscope slides and allowed to dry before adding mounting medium and sealed with coverslips and nail polish.

Image acquisition was performed on Olympus VS 120 at 20X magnification and analyzed with ImageJ software. Image analysis was performed in a user-blinded manner and regions of interests from the infarcted hemispheres were cropped to isolate the ventricular zone (VZ) closest to the stroke. Images from the contralateral hemispheres were taken from a similar area in the VZ of the non-infarcted hemispheres. Sections were removed from analysis if they failed to retain a complete VZ due to stroke. Number of Sox2 positive cells for each image was quantified using Particle Analysis tool of ImageJ and Unpaired t tests (planned comparison) were performed using GraphPad Prism software for statistical analysis.

### Results:

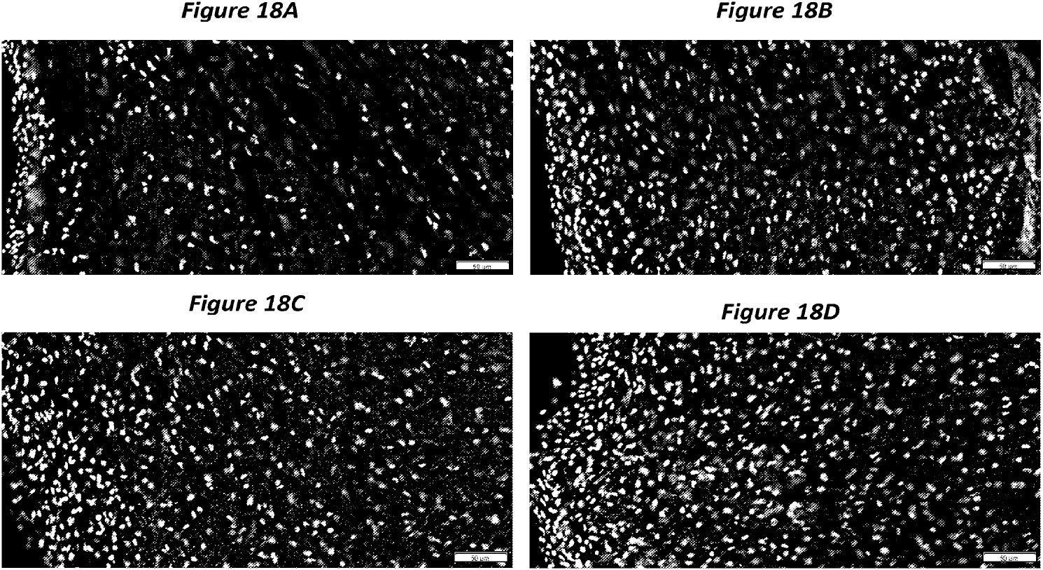

FIGS. 18A - 18D depict a series of images showing replenishment of progenitor cells in the subventricular zone ipsilateral to the injury site for animals sacrificed 29 days post-injury. The different amounts of administration were measured: FIG. 18A (Vehicle), FIG. 18B (1 mg/kg), FIG. 18C (2 mg/kg), and FIG. 18D (4 mg/kg). Just from observation, the scan show substantially increased Sox 2 positive cells at 1 mg/kg, 2 mg/kg, and 4 mg/kg rGDF1 1 administration.

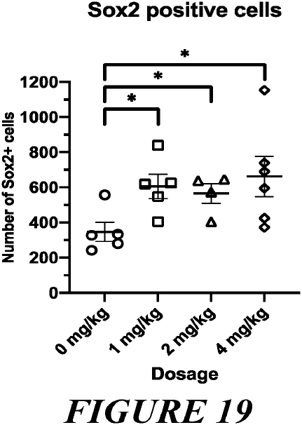

FIG. 19 depicts an analysis of the data of FIGS. 18A - D. Data shown as mean +/- S.E.M., statistically using an unpaired t test * p-value<0.05. Each of the 1 mg/kg dose, 2 mg/kg dose, and 4 mg/kg dose of GDF11 shows statistically significant increase in the number of Sox 2 positive cells. The statistically significant increase in neurogenesis corresponds to the improved behavioral tests for rGDF11 administered mice. Collectively, the data show replenishment of progenitor cells in the subventricular zone ipsilateral to the injury site.

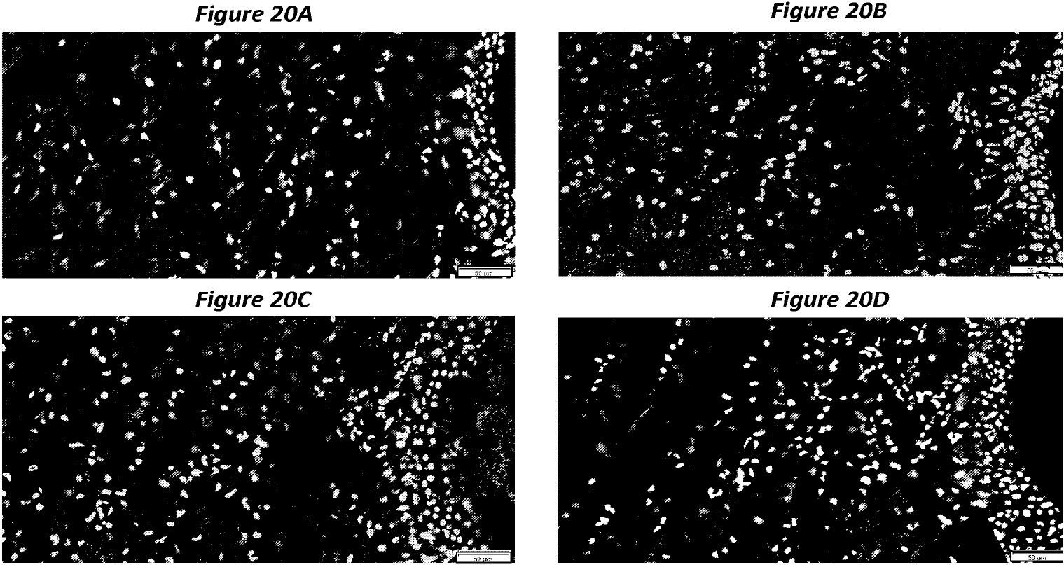

FIGS. 20A - 20D depict replenishment of progenitor cells in the subventricular zone contrallateral to the injury site for animals sacrificed 29 days post-injury. The different amounts of administration were measured: FIG. 20A (Vehicle), FIG. 20B (1 mg/kg), FIG. 20C (2 mg/kg), and FIG. 20D (4 mg/kg). Just from observation, the scan show substantially increased Sox 2 positive cells at 1 mg/kg, 2 mg/kg, and 4 mg/kg rGDF1 1 administration.

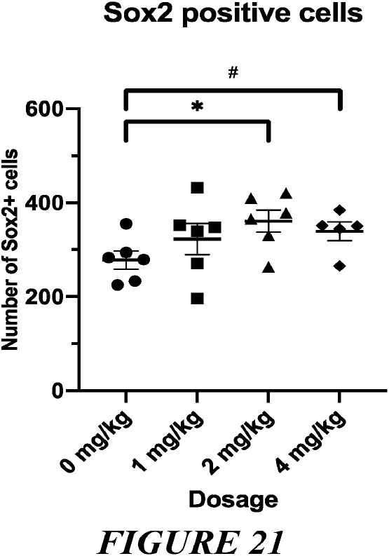

FIG. 21 depicts an analysis of the data of FIGS. 20A - D. Data shown as mean +/- S.E.M., statistically using an unpaired t test * p-value<0.05, # p-value<0.1. Each of the 1 mg/kg dose and 2 mg/kg dose showed a statistically significant (p <0.05). A 4 mg/kg dose of GDF11 had p<0.10 in the number of Sox 2 positive cells for each of the administered groups. The statistically significant increase corresponds to the improved behavioral tests for rGDF11 administered mice. Collectively, the data show replenishment of progenitor cells in the subventricular zone contralateral to the injury site.

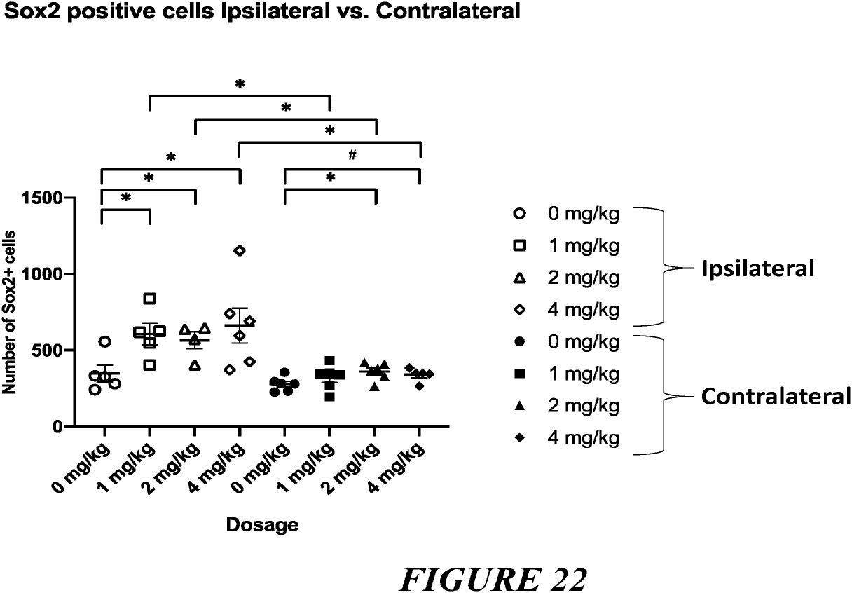

FIG. 22 depicts an analysis comparing hemispheres ipsilateral and contralateral to the injury site. Data shown as mean +/- S.E.M., statistically using an unpaired t test * p-value<0.05, # p-value<0.1. The data show that GDF11 has a greater effect on regeneration of progenitor cells in the SVZ of the infarcted ipsilateral hemisphere than the contralateral hemisphere.

1. A method for treating stroke in a subject, comprising administration of a therapeutically effective amount of a growth differentiation factor 11 (GDF11) molecule to the subject within 12 to 72 hours after a stroke event in the subject, wherein the GDF11 molecule is administered in an amount of at least the minimal high dose of GDF11 relative to the body weight of the subject per day over a treatment period of from 2 to about 14 days.

2. The method of claim 1 wherein administration of the GDF11 molecule is initiated within 12 to 24 hours after the stroke event.

3. The method of claim 1, wherein administration of the GDF11 molecule is carried out over a treatment period of from 2 to 4 days.

4. The method of claim 1, wherein the GDF11 molecule is administered to the subject on a once daily (QD) basis.

5. The method of claim 1, wherein the GDF11 molecule is administered to the subject on intermittent days.

6. The method of claim 1, wherein the GDF11 molecule is a mature form of a GDF11 polypeptide.

7. The method of claim 6 wherein the mature form of the GDF11 polypeptide forms a homodimer.

8. The method of claim 1, wherein the GDF11 molecule is a polypeptide with at least 91% sequence homology with the native sequence of the human GDF11 molecule.

9. The method of claim 8 wherein the GDF11 molecule is recombinant human GDF11 (rhGDF11) comprising SEQ ID NO:1.

10. (canceled)

11. (canceled)

12. The method of claim 1, wherein the GDF11 molecule comprises one or more amino acid substitutions or deletions relative to the native sequence of the human GDF11 molecule.

13. (canceled)

14. (canceled)

15. The method of claim 1, wherein the GDF11 molecule is phosphorylated, glycated, glycosylated, pegylated, HESylated, ELPylated, lipidated, acetylated, amidated, end-capped, includes a cyano group, albumin, or is cyclized.

16. (canceled)

17. (canceled)

18. (canceled)

19. The method of claim 1, wherein the GDF11 molecule is administered to the subject in the form of a pharmaceutical composition comprising a pharmaceutically acceptable carrier, excipient or vehicle.

20. The method of claim 19 wherein the pharmaceutical composition comprises water for injection.

21. The method of claim 20 wherein the pharmaceutical composition is administered to the subject via intravenous injection.

22. The method of claim 1, wherein dosing of the GDF11 molecule is titrated upwards from an initial minimal high dose on day 1 of the administration period to a higher dose on the final day of the administration period.

23. The method of claim 1, wherein dosing of the GDF11 molecule is titrated downwards from an initial high dose to a final minimal high dose on the final day of the administration period.

24. The method of claim 1, further comprising a second administration of a therapeutically effective amount of a GDF11 molecule to the subject, wherein the second administration is carried out after from 2 to 7 days after completion of the initial administration.

25. The method of claim 1, wherein treatment of the subject is characterized by improved body motor or cognitive function in the subj ect.

26. The method of claim 1, wherein the stroke event is ischemic.

27. The method of claim 1, wherein the stroke event is hemorrhagic.

28-46. (canceled)
